(19) United States
(12) Patent Application Publication
(10) Pub. No.: US 2016/0138110 A1
(43) Pub. Date: May 19, 2016

(54) GLIOMA BIOMARKERS

(71) Applicant: Northwestern University, Evanston, IL (US)

(72) Inventors: Robert W. Tell, Chicago, IL (US); Curt M. Horvath, Wilmette, IL (US)

(21) Appl. No.: 14/829,766

(22) Filed: Aug. 19, 2015

Related U.S. Application Data
(60) Provisional application No. 62/039,175, filed on Aug. 19, 2014.

(51) Int. Cl. C12Q 1/68

(52) U.S. Cl. CPC C12Q 1/6886; C12Q 2600/158

(57) ABSTRACT

Provided herein are biomarkers for evaluating malignant glioma.

### CROSS REFERENCE TO RELATED APPLICATIONS

The present application claims the benefit of U.S. Provisional Patent Application Ser. No. 62/039,175, filed Aug. 19, 2014, which is incorporated by reference in its entirety.

### FIELD

Provided herein are biomarkers for evaluating malignant glioma.

### BACKGROUND

Malignant glioma is the most common form of neurological malignancy in the world consisting of 80% of all brain tumors. In the United States, there are 10,000 people newly diagnosed each year (Wen et al., 2008; herein incorporated by reference in its entirety). Malignant glioma is characterized by common p53 and IDH1 mutations along with poor patient outcomes (Jiao et al., 2012; herein incorporated by reference in its entirety). Furthermore, malignant glioma is difficult to treat both surgically and through targeted therapies because of the highly sensitive nature of the surrounding neurological tissue. Furthermore, therapeutic agents must be able to pass the blood-brain barrier or delivered via surgical shunt. This leaves a small selection of agents available for treatment, many of them with significant side effect profiles (Perry et al., 2007; herein incorporated by reference in its entirety). Concomitant with surgical and chemotherapeutic intervention, radiotherapy is often utilized aggressively in these patients (Laperriere et al., 2002; herein incorporated by reference in its entirety). Despite these options, patient mortality remains high, with 60-70% of patients succumbing to the disease within three years of diagnosis. Because of this, it is critical to attempt to lessen the effects of this devastating disease.

Signal transducer and activator of transcription 3 (STAT3) is a latent transcription factor associated with inflammatory signaling and the innate and adaptive immune response (Fagard et al., 2013; herein incorporated by reference in its entirety). In many malignancies, STAT3 is constitutively phosphorylated resulting in overactive signaling (Kamran et al., 2013; herein incorporated by reference in its entirety). This signaling is associated with increased tumor invasion, metastasis, angiogenesis, and drug resistance (Yu et al., 2009; herein incorporated by reference in its entirety). In malignant glioma, STAT3 signaling is thought to drive the transition from low to high grade glioma via a number of malignancy promoting pathways (Dunn et al., 2012; herein incorporated by reference in its entirety). Furthermore, patients with high levels of STAT3 signaling suffer significantly higher mortality than those whose tumors display low levels of STAT3 signaling. Because of this, STAT3 has been analyzed as a potential therapeutic target specifically in malignant glioma (Iwamaru et al., 2007; herein incorporated by reference in its entirety). Though much effort has been spent, there have been few advancements in the realm of STAT3 targeted therapeutics (Yue et al., 2009; herein incorporated by reference in its entirety).

### SUMMARY

In some embodiments, methods are provided for detecting the level of at least one, at least two, at least three, at least four, or all of the target molecules selected from SESN1, GJA9, LOC61436, NUTM2F, and HRK, or any sub-combinations thereof, in a sample from a subject.

In some embodiments, methods are provided for detecting the level of at least one, at least two, at least three, at least four, or at least five glioma biomarkers identified in experiments conducted during development of embodiments of the present invention. In some embodiments, biomarkers are selected from SESN1, GJA9, LOC61436, NUTM2F, and HRK, or any sub-combinations thereof. In some embodiments, a method comprises detecting the level of one or more biomarkers in a sample from a subject.

In some embodiments, a method of monitoring glioma (e.g., response to treatment, likelihood of mortality, etc.) in a subject comprises forming a biomarker panel having N biomarker proteins from glioma biomarkers identified in experiments conducted during development of embodiments of the present invention (e.g., comprising SESN1, GJA9, LOC61436, NUTM2F, and HRK, or any sub-combinations thereof), and detecting the level of each of the N biomarker proteins of the panel in a sample from the subject. In some embodiments, N is 1 to 5. In some embodiments, N is 2 to 4. In some embodiments, methods comprise panels of any combination of the glioma biomarkers identified in experiments conducted during development of embodiments of the present invention (e.g., SESN1, GJA9, LOC61436, NUTM2F, and HRK, or any sub-combinations thereof), in addition to any other glioma or cancer biomarkers.

In some embodiments, methods comprise comparing biomarker(s) level to a reference value/range or a threshold. In some embodiments, deviation of the biomarker(s) level from the reference value/range, or exceeding or failing to meet the threshold, is indicative of a diagnosis, prognosis, etc. for the subject.

In any of the embodiments described herein, each biomarker may be a protein biomarker. In any of the embodiments described herein, the method may comprise contacting biomarkers of the sample from the subject with a set of biomarker capture reagents, wherein each biomarker capture reagent of the set of biomarker capture reagents specifically binds to a biomarker being detected. In some embodiments, each biomarker capture reagent of the set of biomarker capture reagents specifically binds to a different biomarker being detected. In any of the embodiments described herein, each biomarker capture reagent may be an antibody or an aptamer.

In some embodiments, a biomarker is an RNA transcript. In any of the embodiments described herein, the method may comprise contacting biomarkers of the sample from the subject with a set of biomarker capture reagents, wherein each biomarker capture reagent of the set of biomarker capture reagents specifically binds to a biomarker being detected. In some embodiments, each biomarker capture reagent of the set of biomarker capture reagents specifically binds to a different biomarker being detected. In any of the embodiments described herein, each biomarker capture reagent may be a nucleic acid probe.

In any of the embodiments described herein, the sample may be a biological sample (e.g., tissue, fluid (e.g., blood, urine, saliva, etc.), etc.). In some embodiments, the sample is filtered, concentrated (e.g., 2-fold, 5-fold, 10 fold, 20-fold, 50-fold, 100-fold, or more), diluted, or un-manipulated.

In any of the embodiments described herein, a methods further comprise treating the subject for glioma. In some embodiments, treating the subject for glioma comprises a treatment regimen of administering one or more chemotherapeutic, radiation, surgery, etc. In some embodiments, biomarkers described herein are monitored before, during, and/or after treatment.

In some embodiments, methods comprise providing palliative treatment (e.g., symptom relief) to a subject suffering from glioma, but not providing interventional treatment of the glioma. In some embodiments, when embodiments herein indicate a low likelihood of success in treating glioma, palliative care is pursued in place of glioma treatment. In some embodiments, palliative care is provided in addition to treatment for glioma.

In some embodiments, methods of monitoring progression or severity of glioma and/or monitoring effectiveness of treatment in a subject are provided. In some embodiments, a method comprises detecting the level of one or more glioma biomarkers identified in experiments conducted during development of embodiments of the present invention (e.g., SESN1, GJA9, LOC61436, NUTM2F, and HRK, or any sub-combinations thereof) in a sample from the subject at a first time point. In some embodiments, the method further comprises measuring the level one or more of the biomarkers at a second time point. In some embodiments, glioma severity is improving (e.g., declining) if the level of said biomarkers improved at the second time point than at the first time point.

In some embodiments, biomarkers or panels thereof provide a prognosis regarding the future course a glioma in a subject (e.g., likelihood of survival, likelihood of mortality, likelihood of response to therapy, etc.). In some embodiments treatment decisions (e.g., whether to treat, surgery, radiation, chemotherapy, etc.) are made based on the detection and/or quantification of one or more (e.g., 1, 2, 3, 4, 5) of the biomarkers identified in experiments conducted during development of embodiments of the present invention (e.g., comprising SESN1, GJA9, LOC61436, NUTM2F, and HRK, or any sub-combinations thereof).

In some embodiments, kits are provided. In some embodiments, a kit comprises at least one, at least two, at least three, at least four, of at least five capture/detection reagents (e.g., antibody, probe, etc.), wherein each capture/detection reagents specifically binds to a different biomarker (e.g., protein or nucleic acid) selected from the glioma biomarkers identified in experiments conducted during development of embodiments of the present invention (e.g., SESN1, GJA9, LOC61436, NUTM2F, and HRK). In some embodiments, a kit comprises N capture/detection reagents. In some embodiments, N is 1 to 30. In some embodiments, N is 2 to 30. In some embodiments, N is 3 to 30. In some embodiments, N is 4 to 30. In some embodiments, N is 5 to 30. In some embodiments, N is 1 to 10. In some embodiments, N is 2 to 10. In some embodiments, N is 3 to 10. In some embodiments, N is 4 to 10. In some embodiments, N is 5 to 10. In some embodiments, at least one of the N biomarker proteins is selected from the glioma biomarkers identified in experiments conducted during development of embodiments of the present invention (e.g., SESN1, GJA9, LOC61436, NUTM2F, and HRK). In some embodiments, compositions are provided comprising proteins of a sample from a subject and at least one, at least two, at least three, at least four, at least five capture/detection reagents thateach specifically bind to a different biomarker selected from the glioma biomarkers identified in experiments conducted during development of embodiments of the present invention (e.g., SESN1, GJA9, LOC61436, NUTM2F, and HRK).

### BRIEF DESCRIPTION OF THE DRAWINGS

FIGS. 1A-C: Differential mRNA expression analysis of malignant glioma patients. Patients were categorized as STAT3 high or STAT3 low as previously described and mRNA regression analysis was performed utilizing the deSEQ2 R package. A) Kaplan-Meier survival plot comparing patients with high levels of STAT3 (red) to those with normal or low levels of STAT3 (blue). B) Change in expression for all measured genes between the two patient groups. Black indicates the value fell below the FDR adjusted p-value cut of 0.1. Red dots indicate genes above the previously described cut off. C) Heatmap of all tested patients (right axis) comparing those in remission (light gray) with those with active disease (dark gray). Count data were normalized then hierarchically clustered over all genes determined to be differentially expressed in B (bottom axis, left to right, genes shown are: SESN1, CCDC144NL, MT1F, C16ORF89, EGFLAM, DIO3, FOXC2, SELP, CCL13, KCNJ15, NGF, MCTP2, AOC3, APOBEC3A, PDGFRL, EHF, DNAH3, MAPK15, FOXA1, DCDC1, EFCC1, CX3CR1, FAM166A, OR13J1, GJA9, SLC4A9, AMY1A, PAX2, FAM223B, PPP1R27, IL17C, NUTM2F, GJB4, HIST2H3D, HIST1H3J, PCDHA7, CHRNA1, DPY19L2P1, HOXB4, CYP27B1, NPY5R, GSX1, LIN28B, NPSR1, C6ORF123, ELF3, MCOLN3, RMST, LOC340508, GRB7, MYO3A, PAPPA2, GPR31, HMGA2, GAS2, KCNN4, ANKRD2, SEMA4F, RNASE7, HRK, GAST, HTR1F, FGF5, HTR4, KLHL10, LOC641367).

FIGS. 2A-C: A series of feature selection processes detect 9 genes to be highly critical in predicting whether or not patients will be in remission or not at the time of follow up. A) Spike and slab regression analysis was performed as described and the subsequent Bayesian model average (BMA) was ranked against the stability function as determined by the R spikeslab package. The tested genes at each point are as follows: a. SENS1; b. EGFLAM; c. MT1F and C16ORF89, d. LOC641367, SEMA4F, and HRK; e. NUTM2F and GJA9; f. AOC3; g. PAX2, PCDHA7, EHF, HIST1H3D, EFCC1, GPR31, HTR1F, and GAS2; h. NPSR1, PPP1R27, GAST, HOXB4, and ANKRD2; i. CCDC144NL; j. PAPPA2; k. OR13J1; l. GJB4 and IL17C; m. APOBEC3A, NGF, and C6ORF123; n. CYP27B1, SLC4A9 and HIST1H2BF; o. CX3CR1, KLHL10, HMGA2, HIST2H3D, DCDC1, and KCNN4; p. MAPK15, HIST1H3J, DPY19L2P1, and RNASE7; q. KCNJ15, FOXA1, AMY1A, and OPRD1; r. LOC340508; s. PDGFRL, MCTP2, CCL13, SELP, ELF3, CHRNAL FAM223B, FGF5, FOXC2, MYO3A, DIO3, GRB7, HTR4, DNAH3, FAM166A, NPY5R, MCOLN3, RMST, GSX1, and LIN28B. Genes determined to have 100% stability and significant BMA scores were selected for further analysis. B) A similar process was performed utilizing random forest variable selection in order to validate the results of the spike and slab regression analysis. Random forest variable importance ranked the 9 previously detected genes as those with the highest change in out of bag misclassification rate in response to variable permutation. Thus it was determined that these 9 genes are highly critical in prediction of whether or not a patient will respond to treatment. C) Log differential mRNA expression of the average expression levels for each of the 9 genes determined to be highly predictive of patient response.

FIGS. 3A-D: QQ and histogram plots of highly predictive variables and subsequent transformation. A) Histogram of SESN1 normalized count values among all patients analyzed. This graph clearly indicates deviations for normality, making the use of methods reliant on normal distribution unlikely to be effective. B) The same analysis can be shown for SESN1 (and many other genes) when a quantile-quantile (QQ) plot is utilized. Red dotted lines indicate the 95% confidence interval for normal distribution as determined via the Kolmogorov-Smirnov statistic. C) A histogram of data in A following log transformation. A more classic standard normal pattern appears in response to the applied transformation. D) Standard normal distribution of the log transformed data is further confirmed via QQ-plot, indicating all points fall closely within the anticipated cumulative distribution function.

### DEFINITIONS

“Biological sample”, “sample”, and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, and serum), sputum, tears, mucus, nasal washes, nasal aspirate, breath, urine, semen, saliva, peritoneal washings, ascites, cystic fluid, meningeal fluid, amniotic fluid, glandular fluid, lymph fluid, nipple aspirate, bronchial aspirate (e.g., bronchoalveolar lavage), bronchial brushing, synovial fluid, joint aspirate, organ secretions, cells, a cellular extract, and cerebrospinal fluid. This also includes experimentally separated fractions of all of the preceding. For example, a blood sample can be fractionated into serum, plasma, or into fractions containing particular types of blood cells, such as red blood cells or white blood cells (leukocytes). In some embodiments, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Exemplary tissues susceptible to fine needle aspiration include lymph node, lung, lung washes, BAL (bronchoalveolar lavage), thyroid, breast, pancreas, and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner (e.g., filtered, diluted, pooled, fractionated, concentrated, etc.) after being obtained from the individual.

“Target”, “target molecule”, and “analyte” are used interchangeably herein to refer to any molecule of interest that may be present in a biological sample. A “molecule of interest” includes any minor variation of a particular molecule, such as, in the case of a protein, for example, minor variations in amino acid sequence, or gain or loss of modifications including disulfide bond formation, glycosylation, lipidation, acetylation, phosphorylation, or any other manipulation or modification, such as conjugation with a labeling component, which does not substantially alter the identity of the molecule/protein. A “target molecule”, “target”, or “analyte” refers to a set of copies of one type or species of molecule or multi-molecular structure. “Target molecules”, “targets”, and “analytes” refer to more than one type or species of molecule or multi-molecular structure. Exemplary target molecules include proteins, polypeptides, nucleic acids, carbohydrates, lipids, polysaccharides, glycoproteins, hormones, receptors, antigens, antibodies, affybodies, antibody mimics, viruses, pathogens, toxic substances, substrates, metabolites, transition state analogs, cofactors, inhibitors, drugs, dyes, nutrients, growth factors, cells, tissues, and any fragment or portion of any of the foregoing. In some embodiments, a target molecule is a protein, in which case the target molecule may be referred to as a “target protein.”

As used herein, a “capture agent” or “capture reagent” refers to a molecule that binds specifically to a biomarker. A “target protein capture reagent” refers to a molecule that binds specifically to a target protein. Nonlimiting exemplary capture reagents include aptamers, antibodies, adnectins, ankyrins, other antibody mimetics and other protein scaffolds, autoantibodies, chimeras, small molecules, nucleic acids, lectins, ligand-binding receptors, imprinted polymers, avimers, peptidomimetics, hormone receptors, cytokine receptors, synthetic receptors, and modifications and fragments of any of the aforementioned capture reagents.

The term “antibody” refers to full-length antibodies of any species and fragments and derivatives of such antibodies, including Fab fragments, F(ab′)2 fragments, single chain antibodies, Fv fragments, and single chain Fv fragments. The term “antibody” also refers to synthetically-derived antibodies, such as phage display-derived antibodies and fragments, affybodies, nanobodies, etc.

The term “nucleic acid probe” or “probe” refers to a molecule capable of sequence specific hybridization to a nucleic acid, and includes analogs of nucleic acids, as are known in the art, e.g. DNA, RNA, peptide nucleic acids, and the like, and may be double-stranded or single-stranded. Also included are synthetic molecules that mimic nucleic acid molecules in the ability to bind to a designated sequence via hydrogen bonding and other chemical interactions. Such molecules include, for example, those in which peptide linkages substitute for phosphate linkages in the backbone of the molecule.

As used herein, “marker” and “biomarker” are used interchangeably to refer to a target molecule that indicates or is a sign of a normal or abnormal process in an individual or of a disease or other condition in an individual. More specifically, a “marker” or “biomarker” is an anatomic, physiologic, biochemical, or molecular parameter associated with the presence of a specific physiological state or process, whether normal or abnormal, and, if abnormal, whether chronic or acute. Biomarkers are detectable and measurable by a variety of methods including laboratory assays and medical imaging. In some embodiments, a biomarker is a target protein.

As used herein, “biomarker level” and “level” refer to a measurement that is made using any analytical method for detecting the biomarker in a biological sample and that indicates the presence, absence, absolute amount or concentration, relative amount or concentration, titer, a level, an expression level, a ratio of measured levels, or the like, of, for, or corresponding to the biomarker in the biological sample. The exact nature of the “level” depends on the specific design and components of the particular analytical method employed to detect the biomarker.

A “control level” of a target molecule refers to the level of the target molecule in the same sample type from an individual that does not have the disease or condition. A “control level” of a target molecule need not be determined each time the present methods are carried out, and may be a previously determined level that is used as a reference or threshold to determine whether the level in a particular sample is higher or lower than a normal level. In some embodiments, a control level in a method described herein is the level that has been observed in one or more subjects that have glioma that did not lead to mortality (e.g., was responsive to treatment). In some embodiments, a control level in a method described herein is the average or mean level, optionally plus or minus a statistical variation, that has been observed in a plurality of subjects with glioma that did not lead to mortality (e.g., was responsive to treatment).

A “threshold level” of a target molecule refers to the level beyond which (e.g., above or below, depending upon the biomarker) is indicative of or diagnostic for a particular disease or condition (e.g., glioma with a low likelihood of being responsive to treatment). A “threshold level” of a target molecule need not be determined each time the present methods are carried out, and may be a previously determined level that is used as a reference or threshold to determine whether the level in a particular sample is higher or lower than a normal level. In some embodiments, a subject with a biomarker level beyond (e.g., above or below, depending upon the biomarker) a threshold level has a statistically significant likelihood (e.g., 80% confidence, 85% confidence, 90% confidence, 95% confidence, 98% confidence, 99% confidence, 99.9% confidence, etc.) of having glioma that is not responsive to treatments.

As used herein, “individual” and “subject” are used interchangeably to refer to a test subject or patient. The individual can be a mammal or a non-mammal. In various embodiments, the individual is a mammal. A mammalian individual can be a human or non-human. In various embodiments, the individual is a human.

“Diagnose”, “diagnosing”, “diagnosis”, and variations thereof refer to the detection, determination, or recognition of a health status or condition of an individual on the basis of one or more signs, symptoms, data, or other information pertaining to that individual. The health status of an individual can be diagnosed as healthy/normal (e.g., a diagnosis of the absence of a disease or condition) or diagnosed as ill/abnormal (e.g., a diagnosis of the presence, or an assessment of the characteristics, of a disease or condition). The terms “diagnose”, “diagnosing”, “diagnosis”, etc., encompass, with respect to a particular disease or condition, the initial detection of the disease; the characterization or classification of the disease; the detection of the progression, remission, or recurrence of the disease; and the detection of disease response after the administration of a treatment or therapy to the individual.

“Prognose”, “prognosing”, “prognosis”, and variations thereof refer to the prediction of a future course of a disease or condition in an individual who has the disease or condition (e.g., predicting patient survival, predicting the need for organ transplant, etc.), and such terms encompass the evaluation of disease response after the administration of a treatment or therapy to the individual. Example prognoses include likelihood of mortality (e.g., <1%, <5%, <10<, <20%, <30%, <40%, <50%, >50%, >60%, >70%, >80%, >90%, >95%, >99%), likelihood of responsiveness to treatment (e.g., <1%, <5%, <10<, <20%, <30%, <40%, <50%, >50%, >60%, >70%, >80%, >90%, >95%, >99%), likely lifespan (e.g., <1 month, <2 months, <3 month, <6 months, <1 year, 2 years, 3 years, >3 years, etc.).

“Evaluate”, “evaluating”, “evaluation”, and variations thereof encompass both “diagnose” and “prognose” and also encompass determinations or predictions about the future course of a disease or condition in an individual who does not have the disease as well as determinations or predictions regarding the likelihood that a disease or condition will recur in an individual who apparently has been cured of the disease. The term “evaluate” also encompasses assessing an individual's response to a therapy, such as, for example, predicting whether an individual is likely to respond favorably to a therapeutic agent or is unlikely to respond to a therapeutic agent (or will experience toxic or other undesirable side effects, for example), selecting a therapeutic agent for administration to an individual, or monitoring or determining an individual's response to a therapy that has been administered to the individual. Thus, “evaluating” glioma can include, for example, any of the following: diagnosing a subject as having glioma, determining a subject should undergo further testing (e.g., biopsy); prognosing the future course of glioma in an individual; determining whether a treatment being administered is effective in the individual; determining whether an individual will require different treatment than one bring administered, determining whether an individual will recover without treatment (e.g., further treatment); or selecting a treatment to administer to an individual based upon a determination of the biomarker levels derived from the individual's biological sample.

As used herein, “detecting” or “determining” with respect to a biomarker level includes the use of both the instrument used (if used) to observe and record a signal corresponding to a biomarker level and the material/s required to generate that signal. In various embodiments, the level is detected using any suitable method, including fluorescence, chemiluminescence, surface plasmon resonance, surface acoustic waves, mass spectrometry, infrared spectroscopy, Raman spectroscopy, atomic force microscopy, scanning tunneling microscopy, electrochemical or biochemical detection methods, nuclear magnetic resonance, quantum dots, and the like.

“Solid support” refers herein to any substrate having a surface to which molecules may be attached, directly or indirectly, through either covalent or non-covalent bonds. A “solid support” can have a variety of physical formats, which can include, for example, a membrane; a chip (e.g., a protein chip); a slide (e.g., a glass slide or coverslip); a column; a hollow, solid, semi-solid, pore- or cavity-containing particle, such as, for example, a bead; a gel; a fiber, including a fiber optic material; a matrix; and a sample receptacle. Exemplary sample receptacles include sample wells, tubes, capillaries, vials, and any other vessel, groove or indentation capable of holding a sample. A sample receptacle can be contained on a multi-sample platform, such as a microtiter plate, slide, microfluidics device, and the like. A support can be composed of a natural or synthetic material, an organic or inorganic material. The composition of the solid support on which capture reagents are attached generally depends on the method of attachment (e.g., covalent attachment). Other exemplary receptacles include microdroplets and microfluidic controlled or bulk oil/aqueous emulsions within which assays and related manipulations can occur. Suitable solid supports include, for example, plastics, resins, polysaccharides, silica or silica-based materials, functionalized glass, modified silicon, carbon, metals, inorganic glasses, membranes, nylon, natural fibers (such as, for example, silk, wool and cotton), polymers, and the like. The material composing the solid support can include reactive groups such as, for example, carboxy, amino, or hydroxyl groups, which are used for attachment of the capture reagents. Polymeric solid supports can include, e.g., polystyrene, polyethylene glycol tetraphthalate, polyvinyl acetate, polyvinyl chloride, polyvinyl pyrrolidone, polyacrylonitrile, polymethyl methacrylate, polytetrafluoroethylene, butyl rubber, styrenebutadiene rubber, natural rubber, polyethylene, polypropylene, (poly)tetrafluoroethylene, (poly)vinylidenefluoride, polycarbonate, and polymethylpentene. Suitable solid support particles that can be used include, e.g., encoded particles, such as Luminex®-type encoded particles, magnetic particles, and glass particles.

### DETAILED DESCRIPTION

Provided herein are biomarkers for evaluating malignant glioma.

With the genomics revolution, a significant volume of human patient data has become available for analysis and characterization (Gonda et al., 2014; herein incorporated by reference in its entirety). Using such data, an analysis of differential mRNA expression levels between patients who respond to treatment and those who do not was conducted during development of embodiments of the present invention. Supervised learning algorithms employed in order to evaluate differential mRNA and protein expression between patients in remission versus those with active disease, and their potential predictive value as applied to a new group is analyzed. These forms of data analysis have proven highly effective in selecting and categorizing human patient disease subgroups (Tan et al., 2003; herein incorporated by reference in its entirety). Through predictive modeling and feature selection, a set of genes was identified in malignant glioma patients with high levels of STAT3 signaling. In some embodiments, these genes predict patient treatment response with 93% accuracy.

Each of the genes identified during development of embodiments of the present invention, with the exception of the one unknown transcript (LOC61436), have significant previous association with cancer and cancer malignancy. Furthermore, two selected genes, SESN1 and HRK are in families associated with response to ionizing radiation (Sanli et al., 2012; herein incorporated by reference in its entirety), response to chemotherapeutic agents, and pro-apoptotic signaling (Cartron et al., 2012; herein incorporated by reference in its entirety).

Sestrin 1 (SESN1) is part of the growth arrest and DNA damage response GADD family of inducible genes (Velaso Miguel et al., 1999; herein incorporated by reference in its entirety). This gene is activated by p53 as well as a number of other transcription factors in response to cellular stress. Patient data indicate that SESN1 is significantly more highly expressed at the mRNA level in the tumors of patients in remission versus those with active disease. The sestrin genes have been previously shown to inhibitors of mTOR and are critical in the normal cellular response to ionizing radiation (Braunstein et al., 2009; herein incorporated by reference in its entirety). Activation of mTOR has been shown to be important to radio-resistance in a number of different model organisms (Braunstein et al., 2009; herein incorporated by reference in its entirety). Furthermore, a recent study indicates that breast cancer cells are sensitized to ionizing radiation when sestrin 2 is artificially up-regulated (Sanli et al., 2012; herein incorporated by reference in its entirety).

Hirakiri BCL2 interacting protein (HRK) is part of a well-studied family of factors associated with cellular apoptosis or the prevention thereof. Studies of HRK indicate that it is a factor associated with pro-apoptotic signaling in a variety of malignancies (Nakamura et al., 2008; herein incorporated by reference in its entirety). HRK is often silenced via aberrant DNA methylation in prostate cancer while significantly reduced methylation is detectable in normal prostate epithelium (Nakamura et al., 2008; herein incorporated by reference in its entirety). Furthermore, treatment with a number of novel chemotherapeutic compounds have resulted in a concomitant increase in HRK (Chang et al., 2013; herein incorporated by reference in its entirety).

Gap junction protein alpha 9 (GJA9) is the primary connexin found within the nervous system and the human retina (Dobrenis et al., 2005; Sohl et al., 2010; herein incorporated by reference in their entireties). While GJA9 has no known mechanistic association with cancer, it has been shown to be aberrantly hypermethylated in human colorectal cancers as well as a mouse chemically induced model of colorectal cancer (Borinstein et al., 2010; herein incorporated by reference in its entirety).

While little is known about NUTM2F, its family member, NUTM1 is associated with a rare translocation that results in a highly fatal cancer (French et al., 2003; herein incorporated by reference in its entirety). This rare translocation t(15:19)(q13,p13.1) will result in an invariably fatal prognosis when detected in midline organ cancers in young children. The fusion results in replacement of the NUTM1 coding region into the BRD4 gene, leaving only the BRD4 promoter (French et al., 2003; herein incorporated by reference in its entirety). It is thought that this fusion results in blockade of c-fos transcription, blocking cellular differentiation by preventing formation of the AP-1 transcription factor complex (Yan et al., 2011; herein incorporated by reference in its entirety).

Because of these genes (e.g., SESN1, GJA9, LOC61436, NUTM2F, HRK) correlate so strongly with response to treatment, these targets could be analyzed in patients with high levels of STAT3 signaling. In some embodiments, if patients prove to have transcript levels correlated with treatment failure, more aggressive or different chemo- and radiotherapeutic regimes are recommended in order to attempt to circumvent the likelihood of treatment failure.

SESN1, GJA9, LOC61436, NUTM2F, HRK, and/or panels thereof (e.g., alone or with other biomarkers) have utility as glioma biomarker, and are capable of discriminating glioma that is treatable and/or has a low to moderate likelihood of causing mortality from glioma that is unlikely to be responsive to one or more treatments or has a high likelihood of mortality.

In some embodiments, a SESN1, GJA9, LOC61436, NUTM2F, and/or HRK detection/quantification assay is performed along with one or more additional assays in order to evaluate glioma in a subject (e.g., provide a prognosis). In some embodiments, a biomarker panel comprises 2, 3, 4, 5, 6, 7, 8, 9, 10, 11, 12, 13, 14, 15, 16, 17, 18, 19, 20 . . . 30 . . . 40, or more biomarkers. In some embodiments, a biomarker panel comprises fewer than 100 biomarkers (e.g., <100, <90, <80, <70, <60, <50, <40, <30, <20, <10, <5). In some embodiments, the number and identity of biomarkers in a panel are selected based on the sensitivity and specificity for the particular combination of biomarker values. The terms “sensitivity” and “specificity” are used herein with respect to the ability to correctly classify an individual, based on one or more biomarker levels detected in a biological sample. “Sensitivity” indicates the performance of the biomarker(s) with respect to correctly classifying individuals having glioma that is likely nonresponsive to treatment or has a high likelihood of causing mortality. “Specificity” indicates the performance of the biomarker(s) with respect to correctly classifying individuals who do not have glioma that is likely nonresponsive to treatment or has a high likelihood of causing mortality. For example, 85% specificity and 90% sensitivity for a panel of markers used to test a set of control samples and test samples indicates that 85% of the control samples were correctly classified as control samples by the panel, and 90% of the test samples were correctly classified as test samples by the panel.

In some embodiments, methods comprise contacting a sample or a portion of a sample from a subject with at least one detection/capture reagent, wherein each capture reagent specifically binds a biomarker (e.g., protein, nucleic acid, etc.) whose presence and/or level is being detected. In some embodiments, capture reagents are antibodies, aptamers, probes, etc. In some embodiments, a method comprises detecting the level of a first biomarker (or panel of biomarkers) by contacting a sample with detection and/or capture reagents specific for that biomarker and then detection one or more additional biomarkers.

In addition to testing biomarker levels as a stand-alone diagnostic/prognostic test, in some embodiments, biomarker levels are tested in conjunction with other markers or assays that are indicative of a particular glioma diagnosis/prognosis (e.g., imaging, biopsy, etc.). In addition to testing biomarker levels, information regarding the biomarkers may also be evaluated in conjunction with other types of data, particularly data that indicates an individual's risk for glioma (e.g., lifestyle, genetics, age, etc.). These various data can be assessed by automated methods, such as a computer program/software, which can be embodied in a computer or other apparatus/device.

### Detection of Biomarkers and Determination of Biomarker Levels

The presence of a biomarker or a biomarker level for the biomarkers described herein can be detected using any of a variety of analytical methods. In one embodiment, a biomarker level is detected using a capture reagent. In various embodiments, the capture reagent is exposed to the biomarker in solution or is exposed to the biomarker while the capture reagent is immobilized on a solid support. In other embodiments, the capture reagent contains a feature that is reactive with a secondary feature on a solid support. In these embodiments, the capture reagent is exposed to the biomarker in solution, and then the feature on the capture reagent is used in conjunction with the secondary feature on the solid support to immobilize the biomarker on the solid support. The capture reagent is selected based on the type of analysis to be conducted. Capture reagents include but are not limited to aptamers, antibodies, adnectins, ankyrins, other antibody mimetics and other protein scaffolds, autoantibodies, chimeras, small molecules, F(ab′)2 fragments, single chain antibody fragments, Fv fragments, single chain Fv fragments, nucleic acids, lectins, ligand-binding receptors, affybodies, nanobodies, imprinted polymers, avimers, peptidomimetics, hormone receptors, cytokine receptors, and synthetic receptors, and modifications and fragments of these.

In some embodiments, biomarker presence or level is detected using a biomarker/capture reagent complex. In some embodiments, the biomarker presence or level is derived from the biomarker/capture reagent complex and is detected indirectly, such as, for example, as a result of a reaction that is subsequent to the biomarker/capture reagent interaction, but is dependent on the formation of the biomarker/capture reagent complex.

In some embodiments, biomarker presence or level is detected directly from the biomarker in a biological sample.

In some embodiments, biomarkers are detected using a multiplexed format that allows for the simultaneous detection of two or more biomarkers in a biological sample. In some embodiments of the multiplexed format, capture reagents are immobilized, directly or indirectly, covalently or non-covalently, in discrete locations on a solid support. In some embodiments, a multiplexed format uses discrete solid supports where each solid support has a unique capture reagent associated with that solid support, such as, for example quantum dots. In some embodiments, an individual device is used for the detection of each one of multiple biomarkers to be detected in a biological sample. Individual devices are configured to permit each biomarker in the biological sample to be processed simultaneously. For example, a microtiter plate can be used such that each well in the plate is used to analyze one or more of multiple biomarkers to be detected in a biological sample.

In one or more of the foregoing embodiments, a fluorescent tag is used to label a component of the biomarker/capture reagent complex to enable the detection of the biomarker level. In various embodiments, the fluorescent label is conjugated to a capture reagent specific to any of the biomarkers described herein using known techniques, and the fluorescent label is then used to detect the corresponding biomarker level. Suitable fluorescent labels include rare earth chelates, fluorescein and its derivatives, rhodamine and its derivatives, dansyl, allophycocyanin, PBXL-3, Qdot 605, Lissamine, phycoerythrin, Texas Red, and other such compounds.

In some embodiments, the fluorescent label is a fluorescent dye molecule. In some embodiments, the fluorescent dye molecule includes at least one substituted indolium ring system in which the substituent on the 3-carbon of the indolium ring contains a chemically reactive group or a conjugated substance. In some embodiments, the dye molecule includes an AlexFluor molecule, such as, for example, AlexaFluor 488, AlexaFluor 532, AlexaFluor 647, AlexaFluor 680, or AlexaFluor 700. In some embodiments, the dye molecule includes a first type and a second type of dye molecule, such as, e.g., two different AlexaFluor molecules. In some embodiments, the dye molecule includes a first type and a second type of dye molecule, and the two dye molecules have different emission spectra.

Fluorescence can be measured with a variety of instrumentation compatible with a wide range of assay formats. For example, spectrofluorimeters have been designed to analyze microtiter plates, microscope slides, printed arrays, cuvettes, etc. See Principles of Fluorescence Spectroscopy, by J. R. Lakowicz, Springer Science+Business Media, Inc., 2004. See Bioluminescence & Chemiluminescence: Progress & Current Applications; Philip E. Stanley and Larry J. Kricka editors, World Scientific Publishing Company, January 2002.

In one or more embodiments, a chemiluminescence tag is optionally used to label a component of the biomarker/capture complex to enable the detection of a biomarker level. Suitable chemiluminescent materials include any of oxalyl chloride, Rodamin 6G, Ru(bipy)32+, TMAE (tetrakis(dimethylamino)ethylene), Pyrogallol (1,2,3-trihydroxibenzene), Lucigenin, peroxyoxalates, Aryl oxalates, Acridinium esters, dioxetanes, and others.

In some embodiments, the detection method includes an enzyme/substrate combination that generates a detectable signal that corresponds to the biomarker level (e.g., using the techniques of ELISA, Western blotting, isoelectric focusing). Generally, the enzyme catalyzes a chemical alteration of the chromogenic substrate which can be measured using various techniques, including spectrophotometry, fluorescence, and chemiluminescence. Suitable enzymes include, for example, luciferases, luciferin, malate dehydrogenase, urease, horseradish peroxidase (HRPO), alkaline phosphatase, beta-galactosidase, glucoamylase, lysozyme, glucose oxidase, galactose oxidase, and glucose-6-phosphate dehydrogenase, uricase, xanthine oxidase, lactoperoxidase, microperoxidase, and the like.

In some embodiments, the detection method is a combination of fluorescence, chemiluminescence, radionuclide or enzyme/substrate combinations that generate a measurable signal. In some embodiments, multimodal signaling has unique and advantageous characteristics in biomarker assay formats.

In some embodiments, the biomarker levels for the biomarkers described herein is detected using any analytical methods including, singleplex aptamer assays, multiplexed aptamer assays, singleplex or multiplexed immunoassays, mRNA expression profiling, miRNA expression profiling, mass spectrometric analysis, histological/cytological methods, etc. as discussed below.

### Determination of Biomarker Levels Using Immunoassays

Immunoassay methods are based on the reaction of an antibody to its corresponding target or analyte and can detect the analyte in a sample depending on the specific assay format. To improve specificity and sensitivity of an assay method based on immuno-reactivity, monoclonal antibodies and fragments thereof are often used because of their specific epitope recognition. Polyclonal antibodies have also been successfully used in various immunoassays because of their increased affinity for the target as compared to monoclonal antibodies. Immunoassays have been designed for use with a wide range of biological sample matrices. Immunoassay formats have been designed to provide qualitative, semi-quantitative, and quantitative results.

Quantitative results are generated through the use of a standard curve created with known concentrations of the specific analyte to be detected. The response or signal from an unknown sample is plotted onto the standard curve, and a quantity or level corresponding to the target in the unknown sample is established.

Numerous immunoassay formats have been designed. ELISA or EIA can be quantitative for the detection of an analyte. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition; herein incorporated by reference in its entirety).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or for quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Determination of Biomarker Levels Using Gene Expression Profiling

Measuring mRNA in a biological sample may, in some embodiments, be used as a surrogate for detection of the level of a corresponding protein in the biological sample. Thus, in some embodiments, a biomarker or biomarker panel described herein can be detected by detecting the appropriate RNA.

In some embodiments, mRNA expression levels are measured by reverse transcription quantitative polymerase chain reaction (RT-PCR followed with qPCR). RT-PCR is used to create a cDNA from the mRNA. The cDNA may be used in a qPCR assay to produce fluorescence as the DNA amplification process progresses. By comparison to a standard curve, qPCR can produce an absolute measurement such as number of copies of mRNA per cell. Northern blots, microarrays, RNAseq, Invader assays, and RT-PCR combined with capillary electrophoresis have all been used to measure expression levels of mRNA in a sample. See Gene Expression Profiling: Methods and Protocols, Richard A. Shimkets, editor, Humana Press, 2004; herein incorporated by reference in its entirety.

### Detection of Biomarkers Using In Vivo Molecular Imaging Technologies

In some embodiments, a biomarker described herein may be used in molecular imaging tests. For example, an imaging agent can be coupled to a capture reagent, which can be used to detect the biomarker in vivo.

In vivo imaging technologies provide non-invasive methods for determining the state of a particular disease in the body of an individual. For example, entire portions of the body, or even the entire body, may be viewed as a three dimensional image, thereby providing valuable information concerning morphology and structures in the body. Such technologies may be combined with the detection of the biomarkers described herein to provide information concerning the biomarker in vivo.

Advances in the use of in vivo molecular imaging technologies include the development of new contrast agents or labels, such as radiolabels and/or fluorescent labels, which can provide strong signals within the body; and the development of powerful new imaging technology, which can detect and analyze these signals from outside the body, with sufficient sensitivity and accuracy to provide useful information. The contrast agent can be visualized in an appropriate imaging system, thereby providing an image of the portion or portions of the body in which the contrast agent is located. The contrast agent may be bound to or associated with a capture reagent, with a peptide or protein, or an oligonucleotide (for example, for the detection of gene expression), or a complex containing any of these with one or more macromolecules and/or other particulate forms.

The contrast agent may also feature a radioactive atom that is useful in imaging. Suitable radioactive atoms include technetium-99m or iodine-123 for scintigraphic studies. Other readily detectable moieties include, for example, spin labels for magnetic resonance imaging (MM) such as, for example, iodine-123 again, iodine-131, indium-111, fluorine-19, carbon-13, nitrogen-15, oxygen-17, gadolinium, manganese or iron. Such labels are well known in the art and could easily be selected by one of ordinary skill in the art.

Standard imaging techniques include but are not limited to magnetic resonance imaging, computed tomography scanning, positron emission tomography (PET), single photon emission computed tomography (SPECT), and the like. For diagnostic in vivo imaging, the type of detection instrument available is a major factor in selecting a given contrast agent, such as a given radionuclide and the particular biomarker that it is used to target (protein, mRNA, and the like). The radionuclide chosen typically has a type of decay that is detectable by a given type of instrument. Also, when selecting a radionuclide for in vivo diagnosis, its half-life should be long enough to enable detection at the time of maximum uptake by the target tissue but short enough that deleterious radiation of the host is minimized.

Exemplary imaging techniques include but are not limited to PET and SPECT, which are imaging techniques in which a radionuclide is synthetically or locally administered to an individual. The subsequent uptake of the radiotracer is measured over time and used to obtain information about the targeted tissue and the biomarker. Because of the high-energy (gamma-ray) emissions of the specific isotopes employed and the sensitivity and sophistication of the instruments used to detect them, the two-dimensional distribution of radioactivity may be inferred from outside of the body.

Commonly used positron-emitting nuclides in PET include, for example, carbon-11, nitrogen-13, oxygen-15, and fluorine-18. Isotopes that decay by electron capture and/or gamma-emission are used in SPECT and include, for example iodine-123 and technetium-99m. An exemplary method for labeling amino acids with technetium-99m is the reduction of pertechnetate ion in the presence of a chelating precursor to form the labile technetium-99m-precursor complex, which, in turn, reacts with the metal binding group of a bifunctionally modified chemotactic peptide to form a technetium-99m-chemotactic peptide conjugate.

Antibodies are frequently used for such in vivo imaging diagnostic methods. The preparation and use of antibodies for in vivo diagnosis is well known in the art. Similarly, aptamers may be used for such in vivo imaging diagnostic methods. The label used will be selected in accordance with the imaging modality to be used, as previously described. In some embodiments, imaging has unique and advantageous characteristics relating to tissue penetration, tissue distribution, kinetics, elimination, potency, and selectivity as compared to other imaging agents.

Such techniques may also optionally be performed with labeled oligonucleotides, for example, for detection of gene expression through imaging with antisense oligonucleotides. These methods are used for in situ hybridization, for example, with fluorescent molecules or radionuclides as the label. Other methods for detection of gene expression include, for example, detection of the activity of a reporter gene.

Another general type of imaging technology is optical imaging, in which fluorescent signals within the subject are detected by an optical device that is external to the subject. These signals may be due to actual fluorescence and/or to bioluminescence. Improvements in the sensitivity of optical detection devices have increased the usefulness of optical imaging for in vivo diagnostic assays.

Other techniques are review, for example, in N. Blow, Nature Methods, 6, 465-469, 2009; herein incorporated by reference in its entirety.

### Determination of Biomarkers Using Histology/Cytology Methods

In some embodiments, the biomarkers described herein may be detected in a variety of tissue samples using histological or cytological methods. In some embodiments, one or more capture reagent/s specific to the corresponding biomarker/s are used in a cytological evaluation of a sample and may include one or more of the following: collecting a cell sample, fixing the cell sample, dehydrating, clearing, immobilizing the cell sample on a microscope slide, permeabilizing the cell sample, treating for analyte retrieval, staining, destaining, washing, blocking, and reacting with one or more capture reagent/s in a buffered solution. In another embodiment, the cell sample is produced from a cell block.

In some embodiments, one or more capture reagent/s specific to the corresponding biomarkers are used in a histological evaluation of a tissue sample and may include one or more of the following: collecting a tissue specimen, fixing the tissue sample, dehydrating, clearing, immobilizing the tissue sample on a microscope slide, permeabilizing the tissue sample, treating for analyte retrieval, staining, destaining, washing, blocking, rehydrating, and reacting with capture reagent/s in a buffered solution. In another embodiment, fixing and dehydrating are replaced with freezing.

### Determination of Biomarker Levels Using Mass Spectrometry Methods

A variety of configurations of mass spectrometers can be used to detect biomarker levels, and to also define posttranslational modifications (e.g., phosphorylation, glycosylation, acetylation) of the biomarker. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al. Anal. Chem. 70:647 R-716R (1998); Kinter and Sherman, New York (2000): herein incorporated by reference in their entireties).

Protein biomarkers and biomarker levels can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS)N, atmospheric pressure photoionization mass spectrometry (APPI-MS), APPI-MS/MS, and APPI-(MS)N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker levels. Labeling methods include but are not limited to isobaric tag for relative and absolute quantitation (iTRAQ) and stable isotope labeling with amino acids in cell culture (SILAC). In some embodiments, capture reagents are used to selectively enrich samples for candidate biomarker prior to mass spectroscopic analysis.

The foregoing assays enable the detection of biomarker levels that are useful in the methods described herein, where the methods comprise detecting, in a biological sample from an individual, at least one (e.g., CPS1 and/or its post-translationally-modified products), at least two, at least three, at least four, at least five, at least six, at least seven, at least eight, or at least nine biomarkers selected from the described herein or elsewhere. Thus, while some of the described biomarkers may be useful alone for evaluating glioma, methods are also described herein for the grouping of multiple biomarkers and subsets of the biomarkers to form panels of two or more biomarkers. In accordance with any of the methods described herein, biomarker levels can be detected and classified individually or they can be detected and classified collectively, as for example in a multiplex assay format.

### Classification of Biomarkers and Calculation of Disease Scores

In some embodiments, a biomarker “signature” for a given diagnostic or prognostic test contains one or more biomarkers (e.g., a set of markers), each marker having characteristic levels in the populations of interest. Characteristic levels, in some embodiments, may refer to the mean or average of the biomarker levels for the individuals in a particular group. In some embodiments, a diagnostic/prognostic method described herein can be used to assign an unknown sample from an individual into one of two or more groups: high risk glioma, lower risk glioma, treatment-responsive glioma, treatment-unresponsive glioma, healthy, etc. The assignment of a sample into one of two or more groups is known as classification, and the procedure used to accomplish this assignment is known as a classifier or a classification method. Classification methods may also be referred to as scoring methods. There are many classification methods that can be used to construct a diagnostic classifier from a set of biomarker levels. In some instances, classification methods are performed using supervised learning techniques in which a data set is collected using samples obtained from individuals within two (or more, for multiple classification states) distinct groups one wishes to distinguish. Since the class (group or population) to which each sample belongs is known in advance for each sample, the classification method can be trained to give the desired classification response. It is also possible to use unsupervised learning techniques to produce a diagnostic classifier.

Common approaches for developing diagnostic classifiers include decision trees; bagging+boosting+forests; rule inference based learning; Parzen Windows; linear models; logistic; neural network methods; unsupervised clustering; K-means; hierarchical ascending/descending; semi-supervised learning; prototype methods; nearest neighbor; kernel density estimation; support vector machines; hidden Markov models; Boltzmann Learning; and classifiers may be combined either simply or in ways which minimize particular objective functions. For a review, see, e.g., Pattern Classification, R. O. Duda, et al., editors, John Wiley & Sons, 2nd edition, 2001; see also, The Elements of Statistical Learning—Data Mining, Inference, and Prediction, T. Hastie, et al., editors, Springer Science+Business Media, LLC, 2nd edition, 2009.

Exemplary embodiments use any number of the biomarkers provided herein in various combinations (e.g., with other biomarkers of glioma, cancer, general health, etc.) to produce diagnostic/prognostic tests. The markers provided herein can be combined in many ways to produce classifiers.

In some embodiments, once a panel is defined to include a particular set of biomarkers and a classifier is constructed from a set of training data, the diagnostic/prognostic test parameters are complete. In some embodiments, a biological sample is run in one or more assays to produce the relevant quantitative biomarker levels used for classification. The measured biomarker levels are used as input for the classification method that outputs a classification and an optional score for the sample that reflects the confidence of the class assignment.

### Data Analysis and Reporting

In some embodiments, the results are analyzed and/or reported (e.g., to a patient, clinician, researcher, investigator, etc.). Results, analyses, and/or data (e.g., signature, disease score, diagnosis, recommended course, etc.) are identified and/or reported as an outcome/result of an analysis. A result may be produced by receiving or generating data (e.g., test results) and transforming the data to provide an outcome or result. An outcome or result may be determinative of an action to be taken. In some embodiments, results determined by methods described herein can be independently verified by further or repeat testing.

In some embodiments, analysis results are reported (e.g., to a health care professional (e.g., laboratory technician or manager; physician, nurse, or assistant, etc.), patient, researcher, investigator, etc.). In some embodiments, a result is provided on a peripheral, device, or component of an apparatus. For example, sometimes an outcome is provided by a printer or display. In some embodiments, an outcome is reported in the form of a report. Generally, an outcome can be displayed in a suitable format that facilitates downstream use of the reported information. Non-limiting examples of formats suitable for use for reporting and/or displaying data, characteristics, etc. include text, outline, digital data, a graph, graphs, a picture, a pictograph, a chart, a bar graph, a pie graph, a diagram, a flow chart, a scatter plot, a map, a histogram, a density chart, a function graph, a circuit diagram, a block diagram, a bubble map, a constellation diagram, a contour diagram, a cartogram, spider chart, Venn diagram, nomogram, and the like, and combination of the foregoing.

Generating and reporting results from the methods described herein comprises transformation of biological data (e.g., presence or level of biomarkers) into a representation of the characteristics of a subject (e.g., likelihood of mortality, likelihood corresponding to treatment, etc.). Such a representation reflects information not determinable in the absence of the method steps described herein. Converting biologic data into understandable characteristics of a subject allows actions to be taken in response such information.

In some embodiments, a downstream individual (e.g., clinician, patient, etc.), upon receiving or reviewing a report comprising one or more results determined from the analyses provided herein, will take specific steps or actions in response. For example, a decision about whether or not to treat the subject, and/or how to treat the subject is made.

The term “receiving a report” as used herein refers to obtaining, by a communication means, a written and/or graphical representation comprising results or outcomes of analysis. The report may be generated by a computer or by human data entry, and can be communicated using electronic means (e.g., over the internet, via computer, via fax, from one network location to another location at the same or different physical sites), or by another method of sending or receiving data (e.g., mail service, courier service and the like). In some embodiments the outcome is transmitted in a suitable medium, including, without limitation, in verbal, document, or file form. The file may be, for example, but not limited to, an auditory file, a computer readable file, a paper file, a laboratory file or a medical record file. A report may be encrypted to prevent unauthorized viewing.

As noted above, in some embodiments, systems and method described herein transform data from one form into another form (e.g., from biomarker levels to diagnoistic/prognostic determination, etc.). In some embodiments, the terms “transformed”, “transformation”, and grammatical derivations or equivalents thereof, refer to an alteration of data from a physical starting material (e.g., biological sample, etc.) into a digital representation of the physical starting material (e.g., biomarker levels), a condensation/representation of that starting material (e.g., risk level), or a recommended action (e.g., treatment, no treatment, etc.).

### Kits

Any combination of the biomarkers described herein can be detected using a suitable kit, such as for use in performing the methods disclosed herein. The biomarkers described herein may be combined in any suitable combination, or may be combined with other markers not described herein. Furthermore, any kit can contain one or more detectable labels as described herein, such as a fluorescent moiety, etc.

In some embodiments, a kit includes (a) one or more capture reagents for detecting one or more biomarkers in a biological sample, and optionally (b) one or more software or computer program products for providing a diagnosis/prognosis for the individual from whom the biological sample was obtained. Alternatively, rather than one or more computer program products, one or more instructions for manually performing the above steps by a human can be provided.

In some embodiments, a kit comprises a solid support, a capture reagent, and a signal generating material. The kit can also include instructions for using the devices and reagents, handling the sample, and analyzing the data. Further the kit may be used with a computer system or software to analyze and report the result of the analysis of the biological sample.

The kits can also contain one or more reagents (e.g., solubilization buffers, detergents, washes, or buffers) for processing a biological sample. Any of the kits described herein can also include, e.g., buffers, blocking agents, mass spectrometry matrix materials, serum/plasma separators, antibody capture agents, positive control samples, negative control samples, software and information such as protocols, guidance and reference data.

In some embodiments, kits are provided for the analysis of glioma, wherein the kits comprise PCR primers for one or more biomarkers described herein. In some embodiments, a kit may further include instructions for use and correlation of the biomarkers. In some embodiments, a kit may include a DNA array containing the complement of one or more of the biomarkers described herein, reagents, and/or enzymes for amplifying or isolating sample DNA. The kits may include reagents for real-time PCR, for example, TaqMan probes and/or primers, and enzymes.

For example, a kit can comprise (a) reagents comprising at least one capture reagent for determining the level of one or more biomarkers in a test sample, and optionally (b) one or more algorithms or computer programs for performing the steps of comparing the amount of each biomarker quantified in the test sample to one or more predetermined cutoffs. In some embodiments, an algorithm or computer program assigns a score for each biomarker quantified based on said comparison and, in some embodiments, combines the assigned scores for each biomarker quantified to obtain a total score. Further, in some embodiments, an algorithm or computer program compares the total score with a predetermined score, and uses the comparison to determine a diagnosis/prognosis. Alternatively, rather than one or more algorithms or computer programs, one or more instructions for manually performing the above steps by a human can be provided.

### Computer Methods and Software

Once a biomarker or biomarker panel is selected, a method for evaluating glioma in an individual may comprise the following: 1) collect or otherwise obtain a biological sample; 2) perform an analytical method to detect and measure the biomarker or biomarkers in the panel in the biological sample; and 3) report the results of the biomarker levels. In some embodiments, the results of the biomarker levels are reported qualitatively rather than quantitatively, such as, for example, a proposed diagnosis/prognosis or simply a positive/negative result where “positive” and “negative” are defined. In some embodiments, a method for evaluating glioma in an individual may comprise the following: 1) collect or otherwise obtain a biological sample; 2) perform an analytical method to detect and measure the biomarker or biomarkers in the panel in the biological sample; 3) perform any data normalization or standardization; 4) calculate each biomarker level; and 5) report the results of the biomarker levels. In some embodiments, the biomarker levels are combined in some way and a single value for the combined biomarker levels is reported. In this approach, in some embodiments, the reported value may be a single number determined from the sum of all the marker calculations that is compared to a pre-set threshold value that is an indication of the presence or absence of disease. Or the diagnostic score may be a series of bars that each represent a biomarker value and the pattern of the responses may be compared to a pre-set pattern for determination of the presence or absence of disease.

At least some embodiments of the methods described herein can be implemented with the use of a computer. Such a computer may comprise various connected (e.g., directly, wirelessly, over a network, over the web, etc.) or integrated components, including but not limited to: a processor, input device, output device, storage device, computer-readable storage media reader, communications system, processing acceleration (e.g., DSP or special-purpose processors), memory, etc. In some embodiments, computer-readable storage media reader is further coupled to computer-readable storage media, the combination comprehensively representing remote, local, fixed and/or removable storage devices plus storage media, memory, etc. for temporarily and/or more permanently containing computer-readable information, which can include storage device, memory and/or any other such accessible system resource. A system may also comprise software elements including an operating system and other code, such as programs, data and the like.

In one aspect, the system comprises a database containing features of biomarkers characteristic of glioma. The biomarker data (or biomarker information) can be utilized as an input to the computer for use as part of a computer implemented method. The biomarker data can include the data as described herein.

The system may be connectable to a network to which a network server and one or more clients are connected. The network may be a local area network (LAN) or a wide area network (WAN), as is known in the art. Preferably, the server includes the hardware necessary for running computer program products (e.g., software) to access database data for processing user requests.

The system may include one or more devices that comprise a graphical display interface comprising interface elements such as buttons, pull down menus, scroll bars, fields for entering text, and the like as are routinely found in graphical user interfaces known in the art. Requests entered on a user interface can be transmitted to an application program in the system for formatting to search for relevant information in one or more of the system databases. Requests or queries entered by a user may be constructed in any suitable database language. The graphical user interface may be generated by a graphical user interface code as part of the operating system and can be used to input data and/or to display inputted data. The result of processed data can be displayed in the interface, printed on a printer in communication with the system, saved in a memory device, and/or transmitted over the network or can be provided in the form of the computer readable medium.

The system can be in communication with an input device for providing data regarding data elements to the system (e.g., expression values). In one aspect, the input device can include a gene expression profiling system including, e.g., a mass spectrometer, gene chip or array reader, and the like.

The methods and apparatus for analyzing biomarker information according to various embodiments may be implemented in any suitable manner, for example, using a computer program operating on a computer system. A conventional computer system comprising a processor and a random access memory, such as a remotely-accessible application server, network server, personal computer or workstation may be used. Additional computer system components may include memory devices or information storage systems, such as a mass storage system and a user interface, for example a conventional monitor, keyboard and tracking device. The computer system may be a stand-alone system or part of a network of computers including a server and one or more databases.

The biomarker analysis system can provide functions and operations to complete data analysis, such as data gathering, processing, analysis, reporting and/or diagnosis. For example, in one embodiment, the computer system can execute the computer program that may receive, store, search, analyze, and report information relating to the biomarkers. The computer program may comprise multiple modules performing various functions or operations, such as a processing module for processing raw data and generating supplemental data and an analysis module for analyzing raw data and supplemental data to generate a disease status and/or diagnosis. Evaluating glioma in a subject may comprise generating or collecting any other information, including additional biomedical information, regarding the condition of the individual relative to the disease, identifying whether further tests may be desirable, or otherwise evaluating the health status of the individual.

While various embodiments have been described as methods or apparatuses, it should be understood that embodiments can be implemented through code coupled with a computer, e.g., code resident on a computer or accessible by the computer. For example, software and databases could be utilized to implement many of the methods discussed above. Thus, in addition to embodiments accomplished by hardware, it is also noted that these embodiments can be accomplished through the use of an article of manufacture comprised of a computer usable medium having a computer readable program code embodied therein, which causes the enablement of the functions disclosed in this description. Therefore, it is desired that embodiments also be considered protected by this patent in their program code means as well. Furthermore, the embodiments may be embodied as code stored in a computer-readable memory of virtually any kind including, without limitation, RAM, ROM, magnetic media, optical media, or magneto-optical media. Even more generally, the embodiments could be implemented in software, or in hardware, or any combination thereof including, but not limited to, software running on a general purpose processor, microcode, programmable logic arrays (PLAs), or application-specific integrated circuits (ASICs).

### Methods of Treatment

In some embodiments, following a determination that a subject has suffers from glioma, the subject is appropriately treated. In some embodiments, therapy is administered to treat glioma. In some embodiments, therapy is administered to treat complications of glioma (e.g., surgery, radiation, chemotherapy). In some embodiments, treatment comprises palliative care.

In some embodiments, methods of monitoring treatment of glioma are provided. In some embodiments, the present methods of detecting biomarkers are carried out at a time 0. In some embodiments, the method is carried out again at a time 1, and optionally, a time 2, and optionally, a time 3, etc., in order to monitor the progression of glioma or to monitor the effectiveness of one or more treatments of glioma. Time points for detection may be separated by, for example at least 4 hours, at least 8 hours, at least 12 hours, at least 1 day, at least 2 days, at least 4 days, at least 1 week, at least 2 weeks, at least 1 month, at least 2 months, at least 3 months, at least 4 months, at least 6 months, or by 1 year or more. In some embodiments, a treatment regimen is altered based upon the results of monitoring (e.g., upon determining that a first treatment is ineffective). In some embodiments, the level of intervention may be altered.

### EXPERIMENTAL

### Example 1

### Materials and Methods

### Data Collection

All data were downloaded from the TCGA web portal (tcga-data.nci.nih.gov/tcga/). pY-STAT3 levels were determined via the cBioPortal for Cancer Genomics run by the Memorial Sloan Kettering Cancer Center (cbioportal.org/public-portal/index.do) (Cerami et al., 2012; herein incorporated by reference in its entirety). pY-STAT3 levels were plotted for all patients and those with a standard deviation of greater than 0.5 over the mean were selected for analysis as “pY-STAT3 high.” This composed a group of 57 patients after removal of those missing RNA-seq or clinical follow up data. RNA-seq data for all patients displaying gene and raw count values (TCGA level 3) was downloaded and aligned with clinical data based on patient TCGA designation.

### Analysis of RNA Sequencing Data

All downloaded patient sequencing data was collated onto a single spreadsheet utilizing a custom Python harvesting script. Patients were then divided into remission versus active disease and negative binomial regression was employed using the deSEQ2 R package (Anders et al., 2010; herein incorporated by reference in its entirety). DeSEQ2 is specifically designed for analyzing RNA sequencing count data, and as such proves an excellent tool for detecting differentially expressed mRNA transcripts. A cut-off threshold for differential expression was established with the selection of only genes with an FDR-adjusted p-value of 0.1 or less, indicating a likelihood of no greater than 10% chance of being a false positive.

### Feature Selection Via Spike and Slab Regression

Subsequent to the selection of differentially expressed transcripts, feature selection was employed to select for only those transcripts that were putatively predictive of patients being in remission at the time of follow-up. To this end, spike and slab regression analysis was employed (Ishwaran, 2010; herein incorporated by reference in its entirety). This tool utilizes generalized ridge regression computed with a Bayesian model averaging approach. These Bayesian estimates of importance can be utilized for sparse variable selection. Such a tool allows for those genes that are likely to be predictive, as determined by Bayesian model averaging (BMA) to be selected out from the group and analyzed for their predictive capacity through further applications of supervised learning algorithms.

### Feature Selection Via Random Forest Variable Importance Selection

Concomitant with the spike and slab approach, a random forest approach was utilized in order to select variables important to condition classification (Breiman et al., 2001; herein incorporated by reference in its entirety). This process classifies patients based on a random forest classification algorithm then permutes variables at random and assesses changes in the out of bag misclassification rate. In such a way, important features are selected out for their apparent increase in misclassification rate following random permutation.

### Classification of Patients Utilizing Naïve Bayesian Classifier

To classify patients based on differential gene expression, a simple algorithm, specifically naïve Bayes, was utilized. Naïve Bayesian classifiers (NBC) are capable of extremely accurate and precise classification when given a dataset of sufficient size (Caruana et al., 2006; herein incorporated by reference in its entirety). Furthermore, this simple algorithm is highly computationally efficient and can be effectively run in a parallelized fashion. NBC classification was utilized through the e1071 R package (Meyer et al., 2014; herein incorporated by reference in its entirety). The NBC was first trained on a randomly selected training set, then tested for accuracy with the remaining group of patients. Selection of optimally predictive genes was accomplished via parallelized scripting of the NBC wherein genes were discarded at random and classification success was output and recorded. The genes with the highest percentage of successful classifications were selected for further evaluation.

### Classification of Patients Via Support Vector Machine Algorithm

Following selection of highly predictive genes, a more robust learning algorithm useful for comparatively small datasets was employed. This algorithm, the support vector machine (SVM), rapidly classifies data based on weighted covariate values with high accuracy (Meyer et al., 2014; herein incorporated by reference in its entirety). To accomplish this task, the dataset was again split into a training and test set randomly 10 times. Initially, a grid search of gamma and cost function was employed to determine optimal hyperparameter settings. Subsequently, a model was built on the training data and the trained SVM was exposed to the test data. Misclassification rate was analyzed and averaged over a series of ten different trials.

### Example 2

### Results

### Patient Selection

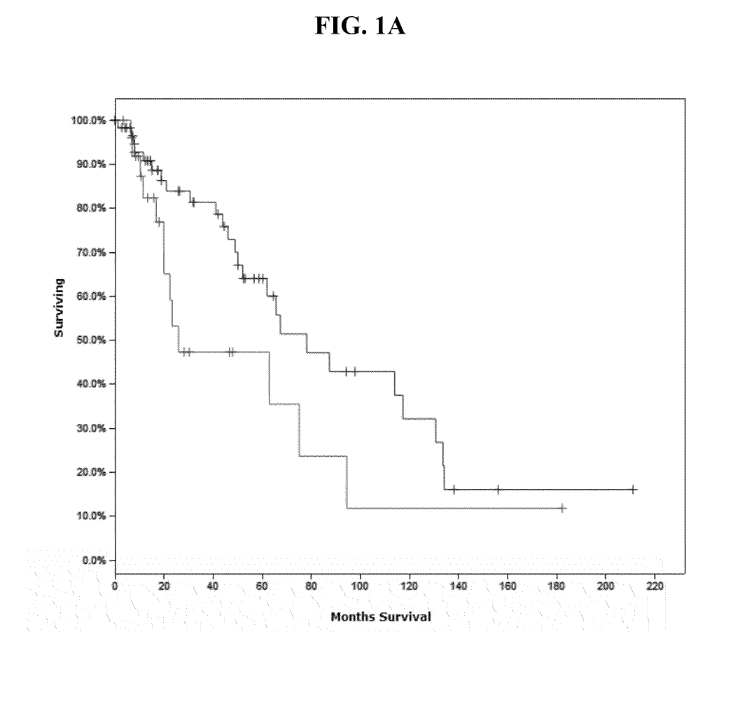

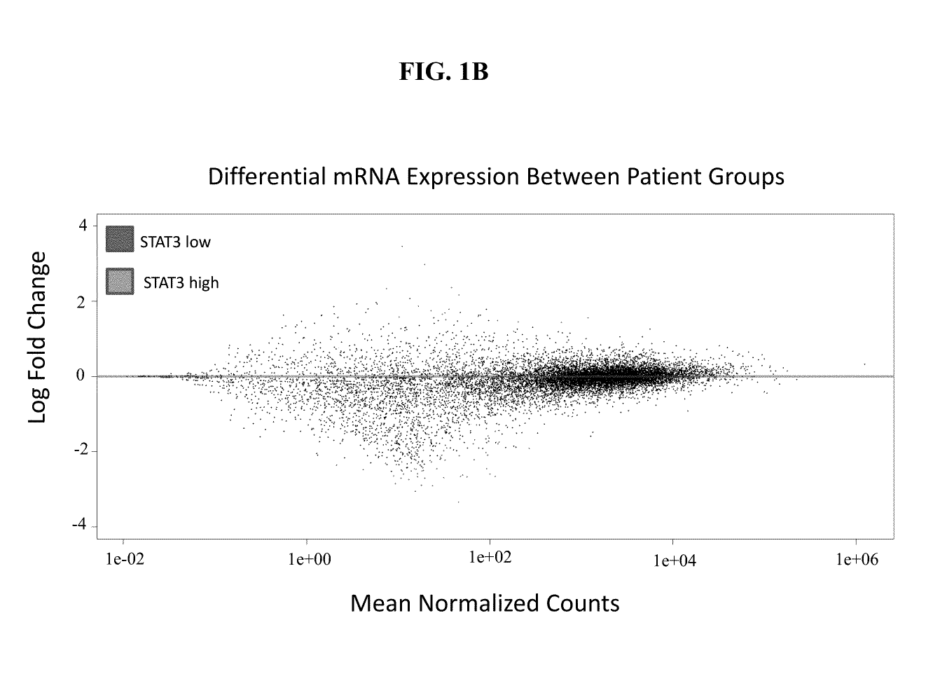

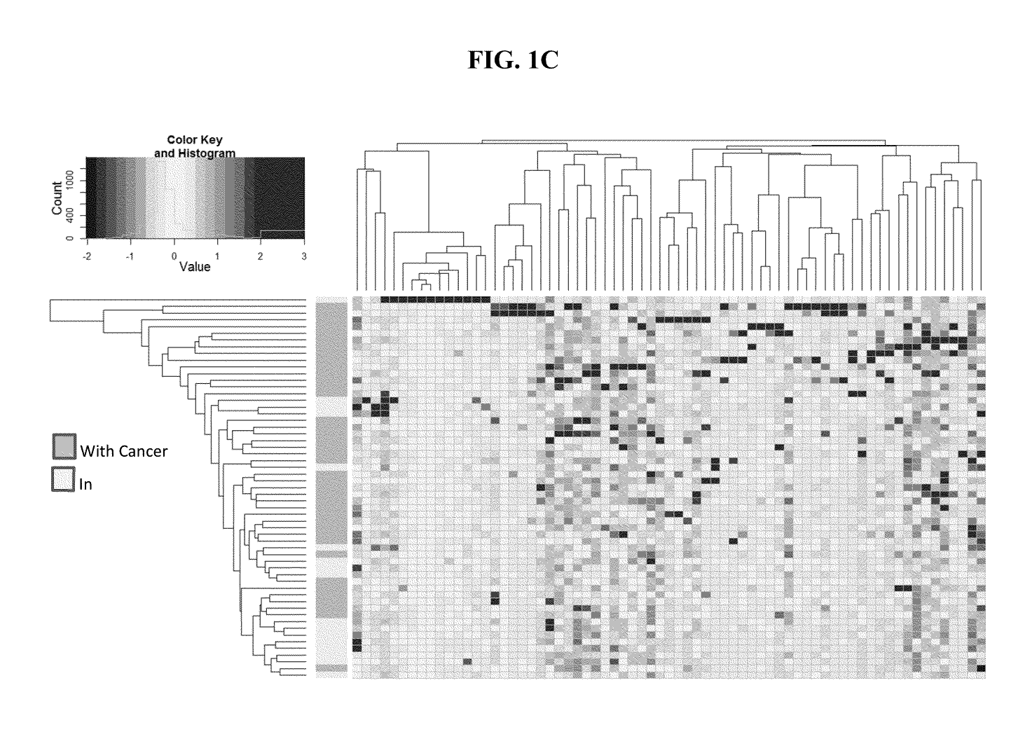

Within the available LGG TCGA dataset of 296 patients, there were a total of 258 patients with RPPA data available. Patients lacking RPPA data were excluded from study as pY-STAT3 levels were unable to be retroactively determined (FIG. 1). Of the patients with available phosphorylation data, only those with high levels of STAT3 were selected. A total of 64 patients with a pY-STAT3 level greater than 0.5 SD above the mean phosphorylation level were selected. Of these patients, 8 were found to have no follow up data and dropped from the study. The remaining patients were divided into two groups, a remission group of 17 patients and a treatment failure group of 40 patients. Patients classified as dead while known to have active disease were placed in the treatment failure group. Of these 57 patients, 37 were found to have received radiotherapy along with chemotherapy and surgery during the course of their treatment while all patients received one treatment or the other along with surgical intervention.

93 Gene Transcripts are Differentially Expressed when Comparing Glioma Patients in Remission Vs Those with Active Disease.

To first determine which genes are differentially expressed, negative binomial regression analysis was utilized via the DeSEQ2 R package (Anders et al., 2013; herein incorporated by reference in its entirety) (FIG. 1). Data for all patients displaying STAT3 tyrosine 705 phosphorylation levels were greater than 0.5 standard deviations from the mean were downloaded along with clinical outcome data from the TCGA portal. This cut-off was selected because patients with high levels of STAT3 signaling have significantly poorer outcomes than patients with lower levels of STAT3 signaling (FIG. 1 A). Patients were placed into one of two groups, one group labeled as currently in remission at follow up, and the second was patients with active disease. All patients selected had to have received an initial glioma diagnosis two years prior to the current study date so as to prevent the inclusion of recently diagnosed patients who had not received the same level of treatment.

Upon analysis, 93 genes were determined to be differentially expressed with an FDR adjusted p-value of less than 0.1 (Benjamin and Hochberg, 1995; herein incorporated by reference in its entirety) (FIG. 1B)(Table 1).

TABLE 1
Differential gene expression analysis comparing STAT3 high patients in remission
versus those with active disease.

| | | | | | FDR |
|---|---|---|---|---|---|
| Gene ID | baseMean | log2FoldChange | SE | pvalue | Adjusted |
| FAM22F\|54754 | 15.27354014 | −3.055565835 | 0.601402 | 3.76E−07 | 0.004414357 |
| FOXA1\|3169 | 45.50696628 | −3.334676101 | 0.676761 | 8.33E−07 | 0.004414357 |
| SELP\|6403 | 19.49003236 | 2.978897216 | 0.609332 | 1.01E−06 | 0.004414357 |
| CCL13\|6357 | 11.05406565 | 3.453783615 | 0.738555 | 2.92E−06 | 0.009526958 |
| NGF\|4803 | 38.07590779 | 2.355854344 | 0.512587 | 4.31E−06 | 0.011242925 |
| NPY5R\|4889 | 50.40142689 | −2.61600576 | 0.577756 | 5.96E−06 | 0.012961735 |
| CHRNA3\|1136 | 45.67544234 | 2.171351917 | 0.487876 | 8.56E−06 | 0.014683649 |
| OR13J1\|392309 | 17.9281695 | −2.891201154 | 0.651184 | 9.00E−06 | 0.014683649 |
| MT1F\|4494 | 1179.972252 | 1.552102847 | 0.354505 | 1.20E−05 | 0.0173532 |
| MCOLN3\|55283 | 56.48251009 | −2.671230939 | 0.618631 | 1.57E−05 | 0.020554792 |
| FGF5\|2250 | 31.05158379 | −2.691638593 | 0.633 | 2.12E−05 | 0.025119633 |
| PCDHA7\|56141 | 267.8110498 | −1.909922782 | 0.451298 | 2.32E−05 | 0.025187295 |
| ELF3\|1999 | 94.78532007 | −2.095975046 | 0.502421 | 3.02E−05 | 0.029249367 |
| GJA9\|81025 | 29.69436972 | −2.093844655 | 0.506653 | 3.59E−05 | 0.029249367 |
| NPSR1\|387129 | 9.284265266 | −2.850313004 | 0.685427 | 3.20E−05 | 0.029249367 |
| SESN1\|27244 | 2782.514405 | 0.909171496 | 0.219436 | 3.42E−05 | 0.029249367 |
| FOXC2\|2303 | 22.72561767 | 1.742537657 | 0.423703 | 3.91E−05 | 0.030034374 |
| GRB7\|2886 | 23.49514888 | −2.903860226 | 0.711565 | 4.49E−05 | 0.032525095 |
| DPY19L2P1\|554236 | 90.74980386 | −2.025670133 | 0.498659 | 4.86E−05 | 0.032816457 |
| HIST1H2BF\|8343 | 19.31025437 | −2.707408868 | 0.667785 | 5.03E−05 | 0.032816457 |
| HIST1H3D\|8351 | 18.63594034 | −1.815437751 | 0.449232 | 5.32E−05 | 0.033054064 |
| EGFLAM\|133584 | 203.4477952 | 1.451329656 | 0.364745 | 6.92E−05 | 0.041056652 |
| CCDC144NL\|339184 | 7.391929136 | 2.337891289 | 0.598346 | 9.34E−05 | 0.048374631 |
| FAM166A\|401565 | 18.59659194 | −2.434389133 | 0.623864 | 9.54E−05 | 0.048374631 |
| KCNN4\|3783 | 125.6191558 | −1.752435157 | 0.450019 | 9.85E−05 | 0.048374631 |
| KLHL10\|317719 | 16.27103605 | −2.3130359 | 0.594543 | 0.0001 | 0.048374631 |
| RNASE7\|84659 | 13.13817414 | −2.736719738 | 0.699911 | 9.23E−05 | 0.048374631 |
| GJB4\|127534 | 13.81868756 | −2.511328741 | 0.648559 | 0.000108 | 0.048553347 |
| IL17C\|27189 | 11.40518554 | −2.591656403 | 0.668557 | 0.000106 | 0.048553347 |
| PAPPA2\|60676 | 122.8646676 | −1.799958541 | 0.466559 | 0.000114 | 0.049751814 |
| DYSFIP1\|116729 | 10.63043466 | −2.706016598 | 0.703925 | 0.000121 | 0.050929936 |
| ANKRD2\|26287 | 32.20367484 | −2.223430594 | 0.580252 | 0.000127 | 0.051881284 |
| GSX1\|219409 | 67.10853262 | −2.210880166 | 0.580053 | 0.000138 | 0.054632095 |
| OPRD1\|4985 | 18.84988062 | −2.49477898 | 0.659534 | 0.000155 | 0.059572896 |
| HIST1H3J\|8356 | 2.116212292 | −2.358205409 | 0.625396 | 0.000163 | 0.060701492 |
| HTR4\|3360 | 28.83507124 | −2.137229346 | 0.573474 | 0.000194 | 0.06178472 |
| KCNJ15\|3772 | 16.55998866 | 1.862568888 | 0.496167 | 0.000174 | 0.06178472 |
| LOC340508\|340508 | 22.23042543 | −2.236329802 | 0.598645 | 0.000187 | 0.06178472 |
| LOC641367\|641367 | 26.06146671 | −1.767686545 | 0.474341 | 0.000194 | 0.06178472 |
| MAPK15\|225689 | 99.5424346 | −2.598888863 | 0.695029 | 0.000185 | 0.06178472 |
| PAX2\|5076 | 26.22659367 | −2.454724314 | 0.655808 | 0.000182 | 0.06178472 |
| GAS2\|2620 | 114.3857995 | −1.457639684 | 0.394497 | 0.00022 | 0.067897062 |
| HTR1F\|3355 | 32.74732678 | −1.82963277 | 0.495746 | 0.000224 | 0.067897062 |
| CCDC48\|79825 | 71.35493142 | −1.290308376 | 0.354926 | 0.000278 | 0.073947833 |
| DCDC1\|341019 | 29.02585981 | −2.179957643 | 0.601208 | 0.000288 | 0.073947833 |
| DNAH3\|55567 | 47.72835031 | −2.657055443 | 0.731803 | 0.000283 | 0.073947833 |
| GAST\|2520 | 16.27691907 | −2.668816813 | 0.73096 | 0.000261 | 0.073947833 |
| HIST2H3D\|653604 | 16.16548986 | −2.054454775 | 0.566414 | 0.000287 | 0.073947833 |
| LIN28B\|389421 | 8.900823004 | −2.649311862 | 0.728422 | 0.000276 | 0.073947833 |
| MCTP2\|55784 | 35.94632437 | 1.787547281 | 0.488459 | 0.000253 | 0.073947833 |
| SEMA4F\|10505 | 664.015444 | −0.674014348 | 0.185933 | 0.000289 | 0.073947833 |
| SLC4A9\|83697 | 13.68433899 | −2.158054221 | 0.599734 | 0.00032 | 0.080386262 |
| EHF\|26298 | 37.85786533 | −1.609053461 | 0.447947 | 0.000328 | 0.080799668 |
| HOXB4\|3214 | 46.50073912 | −2.100890591 | 0.586133 | 0.000338 | 0.081690717 |
| CYP27B1\|1594 | 68.34425207 | −1.682504808 | 0.470918 | 0.000353 | 0.082318651 |
| MYO3A\|53904 | 114.0472052 | −2.652529119 | 0.742351 | 0.000353 | 0.082318651 |
| AOC3\|8639 | 182.9252265 | 1.197150638 | 0.337008 | 0.000382 | 0.082940163 |
| C6orf123\|26238 | 18.25842847 | −2.277348463 | 0.641668 | 0.000387 | 0.082940163 |
| NCRNA00204B\|286967 | 26.1710332 | −1.785325242 | 0.502978 | 0.000386 | 0.082940163 |
| PDGFRL\|5157 | 120.7892954 | 1.484080993 | 0.418241 | 0.000388 | 0.082940163 |
| RMST\|196475 | 96.14619375 | −1.581173181 | 0.445262 | 0.000384 | 0.082940163 |
| C16orf89\|146556 | 603.3419919 | 1.437868872 | 0.407457 | 0.000417 | 0.08646269 |
| LOC157381\|157381 | 18.34402112 | −2.233781255 | 0.632623 | 0.000414 | 0.08646269 |
| AMY1A\|276 | 15.26530954 | −2.014638454 | 0.575128 | 0.00046 | 0.088781709 |
| APOBEC3A\|200315 | 10.19892127 | 1.574985101 | 0.452453 | 0.0005 | 0.088781709 |
| CCRL1\|51554 | 56.9434583 | 1.334431005 | 0.38295 | 0.000493 | 0.088781709 |
| DIO3\|1735 | 30.08204659 | 1.655575026 | 0.476662 | 0.000514 | 0.088781709 |
| GPR31\|2853 | 10.97894757 | −2.515921921 | 0.726846 | 0.000537 | 0.088781709 |
| HMGA2\|8091 | 92.58491721 | −2.4465271 | 0.701881 | 0.000491 | 0.088781709 |
| HRK\|8739 | 21.61395838 | −1.547396713 | 0.446132 | 0.000523 | 0.088781709 |
| LOC440461\|440461 | 18.82079359 | −1.965760599 | 0.566064 | 0.000515 | 0.088781709 |
| NEK10\|152110 | 65.89572887 | −1.798101225 | 0.512407 | 0.00045 | 0.088781709 |
| PAPSS2\|9060 | 1013.688038 | 0.874492066 | 0.252165 | 0.000524 | 0.088781709 |
| PPP3R2\|5535 | 16.21591009 | −2.582037028 | 0.735057 | 0.000444 | 0.088781709 |
| PRDM13\|59336 | 15.69823024 | −2.461755199 | 0.705988 | 0.000489 | 0.088781709 |
| PTPRQ\|374462 | 9.493673972 | −2.349503089 | 0.678617 | 0.000536 | 0.088781709 |
| RAB19\|401409 | 4.136689068 | −2.478602337 | 0.705935 | 0.000446 | 0.088781709 |
| SH3RF2\|153769 | 31.19814742 | −1.969316091 | 0.563679 | 0.000476 | 0.088781709 |
| SLC16A12\|387700 | 32.43347863 | 1.690475924 | 0.48432 | 0.000482 | 0.088781709 |
| ARMS2\|387715 | 14.90431283 | −2.455350991 | 0.714039 | 0.000585 | 0.089948708 |
| CLEC12B\|387837 | 8.346915828 | −1.946785719 | 0.566756 | 0.000593 | 0.089948708 |
| CREM\|1390 | 1273.122662 | 0.703430099 | 0.20398 | 0.000564 | 0.089948708 |
| KCNE1\|3753 | 33.85581894 | −1.897625244 | 0.551659 | 0.000582 | 0.089948708 |
| KRTAP5-8\|57830 | 11.8676917 | −2.118611894 | 0.616554 | 0.00059 | 0.089948708 |
| LOC541473\|541473 | 22.17462587 | −1.842399062 | 0.535446 | 0.00058 | 0.089948708 |
| LRRC14B\|389257 | 10.88486604 | −2.278410752 | 0.661873 | 0.000577 | 0.089948708 |
| SLC30A2\|7780 | 7.222900925 | −2.305990504 | 0.674195 | 0.000625 | 0.093829427 |
| DUOXA2\|405753 | 8.968580465 | −2.117885202 | 0.620276 | 0.000639 | 0.094808385 |
| SLC28A3\|64078 | 18.15840719 | −2.511851629 | 0.736943 | 0.000653 | 0.095810568 |
| ABCA4\|24 | 33.05076788 | 1.50774806 | 0.443491 | 0.000675 | 0.096033303 |
| ADH1B\|125 | 74.72514619 | 1.585235063 | 0.466412 | 0.000677 | 0.096033303 |
| HES2\|54626 | 22.93023161 | −2.269095268 | 0.666909 | 0.000668 | 0.096033303 |

These enriched genes were then hierarchically clustered via complete method and mapped comparing patients with active disease (green) to those currently in remission (yellow). Clustering and heatmap visualization yielded a clearer picture of the differentially expressed gene profiles. A number of selected targets were shown to be biased by significant patient outliers and excluded from further study. Subsequently, gene ontology analysis was employed to look for enrichment of functional groups potentially associated with drug resistance or cancer malignancy (Table 2).

TABLE 2

| GO Term/ | GO Annotation/ | GO Category/ | OT p-value/ | | Corrected | |
|---|---|---|---|---|---|---|
| Gene Name | Gene Description | GeneID | Gene p-value | Enrichment | p-value | 276 |
| GO:0006954 | inflammatory | P | 0.0000331 | 6/266 | 0.0190698 | |
| | response | | | | | |
| AOC3 | amine oxidase, | 8639 | | | | 8639 |
| | copper containing | | | | | |
| | 3 (vascular | | | | | |
| | adhesion protein | | | | | |
| | 1) | | | | | |
| SELP | selectin P (granule | 6403 | | | | 6403 |
| | membrane | | | | | |
| | protein 140 kDa, | | | | | |
| | antigen CD62) | | | | | |
| ELF3 | E74-like factor 3 | 1999 | | | | 1999 |
| | (ets domain | | | | | |
| | transcription | | | | | |
| | factor, epithelial- | | | | | |
| | specific ) | | | | | |
| IL17C | interleukin 17C | 27189 | | | | 27189 |
| NGF | nerve growth | 4803 | | | | 4803 |
| | factor (beta | | | | | |
| | polypeptide) | | | | | |
| CCL13 | chemokine (C-C | 6357 | | | | 6357 |
| | motif) ligand 13 | | | | | |
| GO:0005887 | integral to plasma | C | 0.000415619 | 10/974 | 0.2393968 | |
| | membrane | | | | | |
| KCNJ15 | potassium | 3772 | | | | 3772 |
| | inwardly- | | | | | |
| | rectifying | | | | | |
| | channel, | | | | | |
| | subfamily J, | | | | | |
| | member 15 | | | | | |
| PCDHA7 | protocadherin | 56141 | | | | 56141 |
| | alpha 7 | | | | | |
| NPY5R | neuropeptide Y | 4889 | | | | 4889 |
| | receptor Y5 | | | | | |
| OPRD1 | opioid receptor, | 4985 | | | | 4985 |
| | delta 1 | | | | | |
| GPR31 | G protein-coupled | 2853 | | | | 2853 |
| | receptor 31 | | | | | |
| HTR1F | 5- | 3355 | | | | 3355 |
| | hydroxytryptamine | | | | | |
| | (serotonin) | | | | | |
| | receptor 1F, G | | | | | |
| | protein-coupled | | | | | |
| CX3CR1 | chemokine (C-X3- | 1524 | | | | 1524 |
| | C motif) receptor | | | | | |
| | 1 | | | | | |
| SELP | selectin P (granule | 6403 | | | | 6403 |
| | membrane | | | | | |
| | protein 140 kDa, | | | | | |
| | antigen CD62) | | | | | |
| SEMA4F | sema domain, | 10505 | | | | 10505 |
| | immunoglobulin | | | | | |
| | domain (Ig), | | | | | |
| | transmembrane | | | | | |
| | domain (TM) and | | | | | |
| | short cytoplasmic | | | | | |
| | domain, | | | | | |
| | (semaphorin) 4F | | | | | |
| HTR4 | 5- | 3360 | | | | 3360 |
| | hydroxytryptamine | | | | | |
| | (serotonin) | | | | | |
| | receptor 4, G | | | | | |
| | protein-coupled | | | | | |
| GO:0004930 | G-protein coupled | F | 0.00077307 | 8/728 | 0.4452881 | |
| | receptor activity | | | | | |
| NPSR1 | neuropeptide S | 387129 | | | | 387129 |
| | receptor 1 | | | | | |
| NPY5R | neuropeptide Y | 4889 | | | | 4889 |
| | receptor Y5 | | | | | |
| OPRD1 | opioid receptor, | 4985 | | | | 4985 |
| | delta 1 | | | | | |
| GPR31 | G protein-coupled | 2853 | | | | 2853 |
| | receptor 31 | | | | | |
| HTR1F | 5- | 3355 | | | | 3355 |
| | hydroxytryptamine | | | | | |
| | (serotonin) | | | | | |
| | receptor 1F, G | | | | | |
| | protein-coupled | | | | | |
| CX3CR1 | chemokine (C-X3- | 1524 | | | | 1524 |
| | C motif) receptor | | | | | |
| | 1 | | | | | |
| OR13J1 | olfactory | 392309 | | | | 392309 |
| | receptor, family | | | | | |
| | 13, subfamily J, | | | | | |
| | member 1 | | | | | |
| HTR4 | 5- | 3360 | | | | 3360 |
| | hydroxytryptamine | | | | | |
| | (serotonin) | | | | | |
| | receptor 4, G | | | | | |
| | protein-coupled | | | | | |
| GO:0007186 | G-protein coupled | P | 0.000825162 | 9/892 | 0.4752935 | |
| | receptor protein | | | | | |
| | signaling pathway | | | | | |
| NPSR1 | neuropeptide S | 387129 | | | | 387129 |
| | receptor 1 | | | | | |
| NPY5R | neuropeptide Y | 4889 | | | | 4889 |
| | receptor Y5 | | | | | |
| OPRD1 | opioid receptor, | 4985 | | | | 4985 |
| | delta 1 | | | | | |
| GPR31 | G protein-coupled | 2853 | | | | 2853 |
| | receptor 31 | | | | | |
| HTR1F | 5- | 3355 | | | | 3355 |
| | hydroxytryptamine | | | | | |
| | (serotonin) | | | | | |
| | receptor 1F, G | | | | | |
| | protein-coupled | | | | | |
| CX3CR1 | chemokine (C-X3- | 1524 | | | | 1524 |
| | C motif) receptor | | | | | |
| | 1 | | | | | |
| GAST | gastrin | 2520 | | | | 2520 |
| OR13J1 | olfactory | 392309 | | | | 392309 |
| | receptor, family | | | | | |
| | 13, subfamily J, | | | | | |
| | member 1 | | | | | |
| HTR4 | 5- | 3360 | | | | 3360 |
| | hydroxytryptamine | | | | | |
| | (serotonin) | | | | | |
| | receptor 4, G | | | | | |
| | protein-coupled | | | | | |
| GO:0007267 | cell-cell signaling | P | 0.001398125 | 4/242 | 0.8053203 | |
| FGF5 | fibroblast growth | 2250 | | | | 2250 |
| | factor 5 | | | | | |
| SEMA4F | sema domain, | 10505 | | | | 10505 |
| | immunoglobulin | | | | | |
| | domain (Ig), | | | | | |
| | transmembrane | | | | | |
| | domain (TM) and | | | | | |
| | short cytoplasmic | | | | | |
| | domain, | | | | | |
| | (semaphorin) 4F | | | | | |
| IL17C | interleukin 17C | 27189 | | | | 27189 |
| CCL13 | chemokine (C-C | 6357 | | | | 6357 |
| | motif) ligand 13 | | | | | |

The primary enriched functional group revolved around inflammation with a corrected p-value of 0.027. Because inflammatory processes are strongly associated with response to chemotherapeutic and radiotherapeutic interventions (De Visser et al., 2009; herein incorporated by reference in its entirety), enrichment of inflammatory genes proved a quite promising initial indicator. The next step taken was to utilize feature selection tools in order to select differentially enriched genes whose expression is closely associated with different patient outcomes.

### Spike and Slab Regression and Covariate Stability Analysis Results in 9 Genes Whose Expression Levels are Critically Linked to Treatment Failure.

While predictive modeling can handle a large number of covariates, a more robust model was desired wherein only a small pool of genes might predict patient outcome. It was also critical to determine which genes were passengers and which were drivers of this process, with the goal of detecting genes that may affect outcome rather than simply be associated with it. To this end, feature selection via spike and slab regression was employed (Ishwaran et al., 2005; herein incorporated by reference in its entirety).

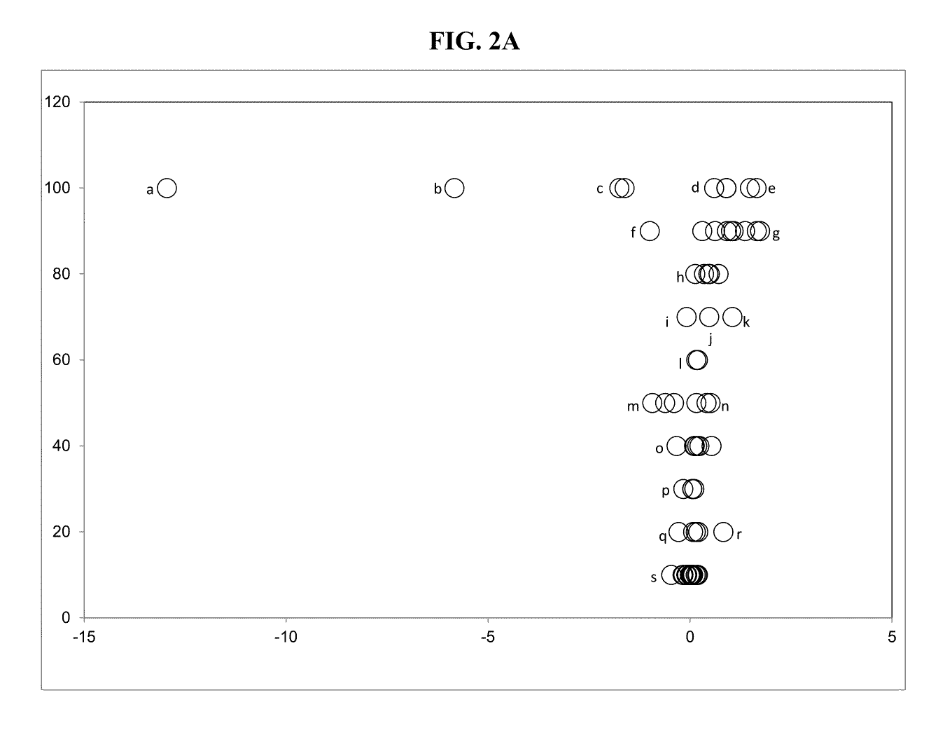

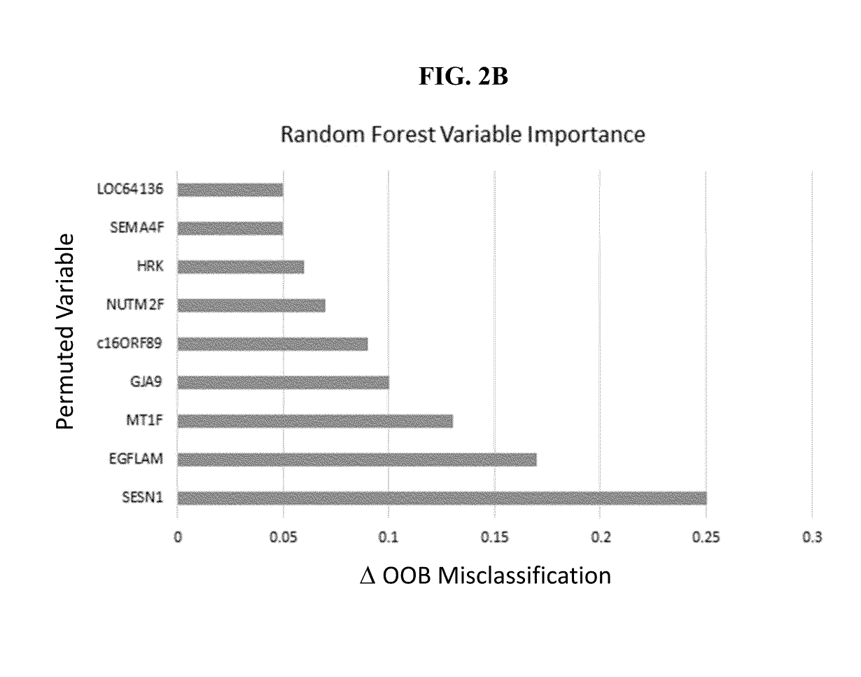

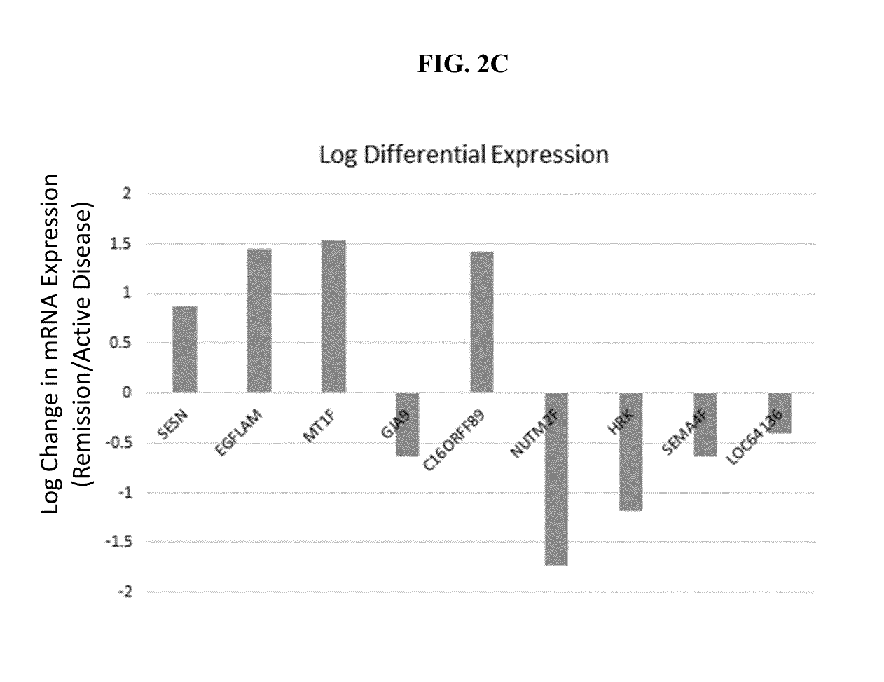

Initially, dummy variables were assigned to each patient outcome, 1 for active disease, and 0 for remission. This was done because the R spikeslab package will not take categorical or factor values and requires numerical input as the response variable. Following regression analysis, covariates were plotted against their cross-validated stability values (FIG. 2 A). 9 genes display 100% stability, indicating significant predictive capacity when attempting to determine whether a patient will be in remission or not at the time of follow-up.

Concomitantly, random forest variable importance analysis was utilized in order to target the feature selection problem from multiple angles. Because the spike and slab approach required the use of dummy variables, a more standard classification algorithm was sought in order to confirm the findings from the regression analysis. Variable permutation was done with all selected genes and the top 9 ranked genes for importance to predictive capacity were the same genes found via the spike and slab regression process (FIG. 2 A, B, C). Following analysis, the same 9 genes (FIG. 2) were determined to be most important for accurate classification of remission. These genes were then selected for further predictive classification via supervised learning algorithm.

Reiterative Prediction with Naïve Bayesian Classification Task Selects for Five Specific Genes with an 89% Capacity to Successfully Predict Patient Remission.

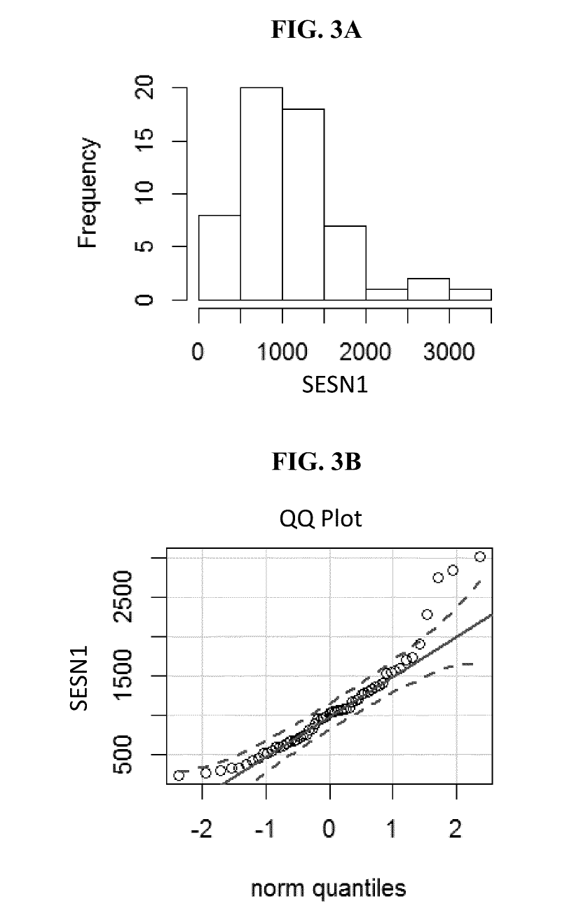

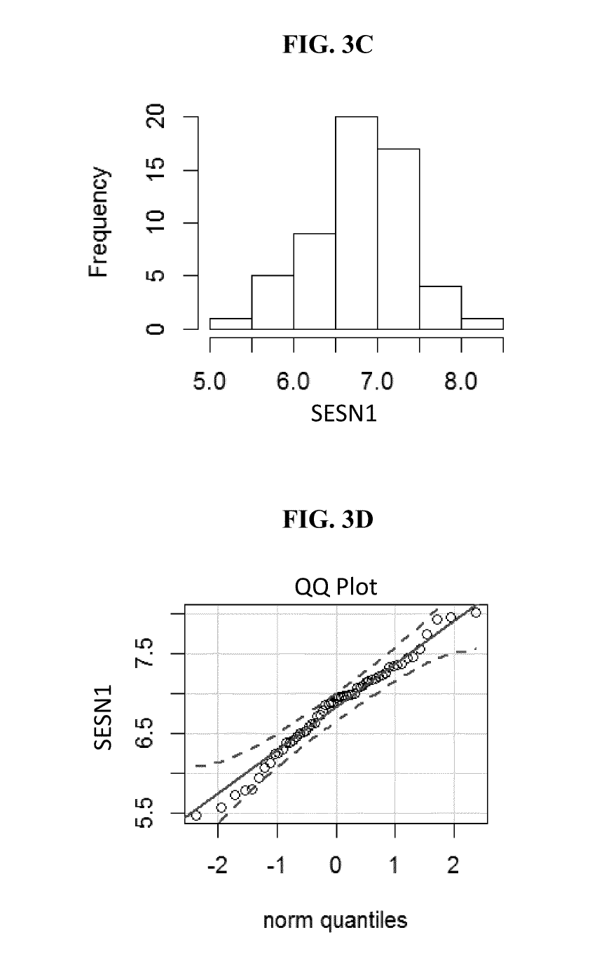

Following the feature selection process, an attempt was made to determine which of the genes could most effectively predict patient outcome via processive analysis. A simple classification algorithm, specifically naïve Bayes, was used on the list of genes, each time splitting the list in half randomly and determining whether classification accuracy improved or diminished with each half. Because a central assumption of the naïve Bayesian classifier is that the data are normally distributed, the data were plotted both via standard histogram and quantile quantile plot (QQ plot) (FIG. 3 A). All analyzed genes followed a likely non-normal distribution with significant positive skew (Lilliefors <0.05); (FIG. 3 A, B). Thus, data were subjected to log transformation and re-plotted (FIG. 3 A) to confirm a normal distribution had been achieved and an NBC could be applied. A single five gene group (SESN1, GJA9, LOC61436, NUTM2F, HRK) were found to have the highest predictive accuracy utilizing NBC. Any attempt to further reduce the number of analyzed genes resulted in a drop of predictive capacity, indicating that these specific genes are the optimal predictors.

Subsequent to selection with a simple classification algorithm that can be effectively parallelized, it was determined that a more robust algorithm may increase overall predictive capacity.

### Support Vector Machine Based Classification of the Five Highly Predictive Genes Results in 93.6% Predictive Accuracy.

Subsequent to selection of the most critically important genes to predictive accuracy, a support vector machine (SVM) algorithm was utilized with the original untransformed values in order to attempt to improve predictive performance. The data for the five genes were split several times into training and test sets via random selection. The SVM was then optimized via grid search of gamma and cost functions on the training data. Furthermore, a weight was added to the training data for misclassification of remission due to the unequal distribution of the dataset between remission vs active disease. Following model generation, the test set was inputted into the trained SVM over a series of different training and test sets. With this process, predictive accuracy over several iterations was as high as 93.6% with a mean over all tests of 92.3%. This indicates as much as a 4% improvement in accuracy with an average of 3.1% over all trials. This displays a significant predictive accuracy in the high-risk, pY-STAT3 high subgroup of human malignant glioma patients based on five specific genes. Some of these genes are associated with both drug and radiation resistance, as well as cell cycle and tumor processes (Table 1).

All publications and patents mentioned in the present application and/or listed below are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

### REFERENCES

Benjamini, Y. and Y. Hochberg (1995). “Controlling the false discovery rate: a practical and powerful approach to multiple testing.” Journal of the Royal Statistical Society. Series B (Methodological): 289-300.
Borinstein, S. C., et al. (2010). “Aberrant DNA methylation occurs in colon neoplasms arising in the azoxymethane colon cancer model.” Mol Carcinog 49(1): 94-103.
Braunstein, S., et al. (2009). “Regulation of protein synthesis by ionizing radiation.” Mol Cell Biol 29(21): 5645-5656.
Breiman, L. (2001). “Random Forests.” Machine Learning 45(1): 5-32.
Cartron, P. F., et al. (2012). “Prognostic impact of the expression/phosphorylation of the BH3-only proteins of the BCL-2 family in glioblastoma multiforme.” Cell Death Dis 3: e421.
Caruana, R. and A. Niculescu-Mizil (2006). An empirical comparison of supervised learning algorithms. Proceedings of the 23rd international conference on Machine learning, ACM.
Chang, I., et al. (2013). “Hrk mediates 2-methoxyestradiol-induced mitochondrial apoptotic signaling in prostate cancer cells.” Mol Cancer Ther 12(6): 1049-1059.
de Visser, K. E. and J. Jonkers (2009). “Towards understanding the role of cancer-associated inflammation in chemoresistance.” Curr Pharm Des 15(16): 1844-1853.
Dobrenis, K., et al. (2005). “Human and mouse microglia express connexin36, and functional gap junctions are formed between rodent microglia and neurons.” J Neurosci Res 82(3): 306-315.
Fagard, R., et al. (2013). “STAT3 inhibitors for cancer therapy: Have all roads been explored?” Jakstat 2(1): e22882.
French, C. A., et al. (2003). “BRD4-NUT fusion oncogene: a novel mechanism in aggressive carcinoma.” Cancer Res 63(2): 304-307.
Gonda, D. D., et al. (2014). “The Cancer Genome Atlas expression profiles of low-grade gliomas.” Neurosurg Focus 36(4): E23.
Ishwaran, H., et al. “spikeslab: Prediction and variable selection using spike and slab regression. The R Journal Vol. 2/2 Dec. 2010
Iwamaru, A., et al. (2007). “A novel inhibitor of the STAT3 pathway induces apoptosis in malignant glioma cells both in vitro and in vivo.” Oncogene 26(17): 2435-2444.
Jiao, Y., et al. (2012). “Frequent ATRX, CIC, FUBP1 and IDH1 mutations refine the classification of malignant gliomas.” Oncotarget 3(7): 709-722.
Kamran, M. Z., et al. (2013). “Role of STAT3 in cancer metastasis and translational advances.” Biomed Res Int 2013: 421821.
Laperriere, N., et al. (2002). “Radiotherapy for newly diagnosed malignant glioma in adults: a systematic review.” Radiother Oncol 64(3): 259-273.
Nakamura, M., et al. (2008). “The role of HRK gene in human cancer.” Oncogene 27 Suppl 1: S105-113.
Sanli, T., et al. (2012). “Sestrin2 modulates AMPK subunit expression and its response to ionizing radiation in breast cancer cells.” PloS one 7(2): e32035.
Tan, A. C. and D. Gilbert (2003). “Ensemble machine learning on gene expression data for cancer classification.” Appl Bioinformatics 2(3 Suppl): S75-83.
Velasco-Miguel, S., et al. (1999). “PA26, a novel target of the p53 tumor suppressor and member of the GADD family of DNA damage and growth arrest inducible genes.” Oncogene 18(1): 127-137.
Wen, P. Y. and S. Kesari (2008). “Malignant gliomas in adults.” N Engl J Med 359(5): 492-507.
Yan, J., et al. (2011). “Perturbation of BRD4 protein function by BRD4-NUT protein abrogates cellular differentiation in NUT midline carcinoma.” J Biol Chem 286(31): 27663-27675.
Burlingame et al. Anal. Chem. 70:647 R-716R (1998).
Kinter and Sherman, New York (2000).
Pattern Classification, R. O. Duda, et al., editors, John Wiley & Sons, 2nd edition, (2001).
The Elements of Statistical Learning—Data Mining, Inference, and Prediction, T. Hastie, et al., editors, Springer Science+Business Media, LLC, 2nd edition, (2009).
Sheila Perry, Theresa L Kowalski, and Chih-Hung Chang, Quality of life assessment in women with breast cancer: benefits, acceptability and utilization, Health and Quality of Life Outcomes 2007, 5:24 (2007).
Hua Yu, Drew Pardoll & Richard Jove, STATs in cancer inflammation and immunity: a leading role for STAT3, Nature Reviews Cancer 9, 798-809 (November 2009).
Gavin P. Dunn, Mikael L. Rinne, Jill Wykosky, Giannicola Genovese, Steven N. Quayle, Ian F. Dunn, Pankaj K. Agarwalla, Milan G. Chheda, Benito Campos, Alan Wang, Cameron Brennan, Keith L. Ligon, Frank Furnari, Webster K. Cavenee, Ronald A. Depinho, Lynda Chin, and William C. Hahn, Emerging insights into the molecular and cellular basis of glioblastoma, Genes Dev. 26: 756-784 (Apr. 15, 2012).
Goran Söhl, Antonia Joussen, Norbert Kociok, and Klaus Willecke, Expression of connexin genes in the human retina, BMC Ophthalmology 2010, 10:27, available at http://www.biomedcentral.com/1471-2415/10/27.
Nathan Blow, In vivo molecular imaging: the inside job, Nature Methods, 6, 465-469, (2009).
Ethan Cerami et al., The cBio Cancer Genomics Portal: An Open Platform for Exploring Multidimensional Cancer Genomics Data, Cancer Discov; 2(5); 401-404 (2012).
Anders S & Huber W (2010) Differential expression analysis for sequence count data. Genome biology 11(10):R106.
Hemant Ishwaran and J. Sunil Rao, Spike and Slab Variable Selection: Frequentist and Bayesian Strategies, Annals of Statistics 33:730-773 (2005).

1-20. (canceled)

21. A method, comprising detecting the level of two or more target analytes, but fewer than 100 target analytes, in a sample, two or more of the target analytes being selected from the group consisting of SESN1, GJA9, LOC61436, NUTM2F.

22. The method of claim 21, further comprising detecting one or more additional target analytes.

23. The method of claim 22, comprising detecting three or more target analytes being selected from the group consisting of SESN1, GJA9, LOC61436, NUTM2F.

24. The method of claim 22, comprising detecting ten or more target analytes.

25. (canceled)

26. The method of claim 21, wherein the sample is a blood product selected from whole blood; plasma; serum; and filtered, concentrated, fractionated or diluted samples of the preceding.

27. The method of claim 21, wherein the method comprises contacting the sample with a set of capture reagents, wherein each capture reagent specifically binds to a different target analyte being detected.

28. The method of claim 27, wherein each capture reagent is an antibody.

29. The method of claim 27, wherein each capture reagent is a nucleic acid probe.

30. Reagents comprising capture reagents for the detection of two or more target analytes, but fewer than 100 target analytes, two or more of the target analytes being selected from the group consisting of SESN1, GJA9, LOC61436, NUTM2F, and HRK.

31. The reagents of claim 30, wherein said capture reagents are antibodies.

31. The reagents of claim 30, wherein said capture reagents are nucleic acid probes.

32. A kit comprising the reagents of claim 30 and one or more additional reagents for carrying out an assay in a sample from a subject.

33. The reagents of claim 30, comprising capture reagents for detecting three or more target analytes selected from the group consisting of SESN1, GJA9, LOC61436, NUTM2F.
